(19) United States
(12) Patent Application Publication
(10) Pub. No.: US 2026/0306189 A1
(43) Pub. Date: Oct. 8, 2026

(54) SYSTEM AND METHOD FOR DIRECT AUGMENTED REALITY MEDICAL REGISTRATION

(71) Applicant: MEDIVIEW XR, INC., Cleveland, OH (US)

(72) Inventors: Seyedsina Shirinpour, Minneapolis, MN (US); Peter Nicholas Braido, Wyoming, MN (US); Ross Daniel Hinrichsen, Plymouth, MN (US); Jeff Yanof, Solon, OH (US)

(21) Appl. No.: 19/639,558

(22) Filed: Apr. 6, 2026

Related U.S. Application Data
(60) Provisional application No. 63/783,480, filed on Apr. 4, 2025.

(51) Int. Cl. A61B 34/20; A61B 34/10; A61B 90/00; A61B 90/50; G06F 3/01; G06T 7/13; G06T 7/246; G06T 7/33; G06T 7/50; G06T 7/73; G06T 15/04; G06T 19/00; G06T 19/20; G16H 30/40

(52) U.S. Cl. CPC A61B 34/20; G06F 3/012; G06T 7/13; G06T 7/246; G06T 7/337; G06T 7/50; G06T 7/73; G06T 15/04; G06T 19/006; G06T 19/20; G16H 30/40; A61B 2034/105; A61B 2034/107; A61B 2034/2063; A61B 2034/2065; A61B 2090/365; A61B 2090/378; A61B 2090/3983; A61B 2090/502; G06T 2200/04; G06T 2207/10132; G06T 2207/20221; G06T 2207/30088; G06T 2207/30204; G06T 2210/41; G06T 2219/2004; G06T 2219/2012

(57) ABSTRACT

Systems and methods for augmented reality medical guidance using multi-stage registration, multi-modal visualization, and dynamic compensation are disclosed. A multi-stage registration method establishes coordinate transformations between a tracking system, an augmented reality system, and a patient's anatomy through device-level registration and anatomical registration. Multiple registration approaches are supported including manual, automated, optical, stylus-based, fiducial-based, surface-based, ultrasound-based, and landmark-based registration. A multi-modal visualization method fuses real-time ultrasound imaging with medical imaging data, displaying holographic content including ultrasound flashlight visualization, texture overlays, edge map fusion, and comprehensive guidance information such as holographic light rays and multi-planar reconstruction slices.

### CROSS-REFERENCE TO RELATED APPLICATIONS

This application claims the benefit of U.S. Provisional Application No. 63/783,480, filed on Apr. 4, 2025. The entire disclosure of the above application is incorporated herein by reference.

### FIELD

The present invention relates to medical imaging and surgical navigation systems, and more particularly to systems and methods for registering and tracking medical devices.

### INTRODUCTION

This section provides background information related to the present disclosure which is not necessarily prior art.

Medical procedures increasingly rely on advanced imaging and visualization technologies to guide complex interventions. Surgical navigation systems can use electromagnetic tracking and optical markers to register medical devices and imaging systems. However, these approaches can require multiple external registration devices and fiducial markers that add complexity, cost, and time to procedural workflows.

Certain registration methods can also face challenges in maintaining continuous alignment throughout medical procedures. When using registration devices, the tracking accuracy can be compromised by interference, line-of-sight limitations, and patient movement. Additionally, systems can depend on a single tracking modality, making them vulnerable to disruption if that primary tracking method becomes temporarily unavailable.

The ergonomics and efficiency of surgical procedures can be impacted by the particular registration approach. Operating rooms face increasing equipment crowding, with multiple devices, tubes, and cables creating physical hazards and decreasing the efficiency of instrument handling. There are issues with cable management and various tripping hazards. The use of ceiling-mounted systems and other registration hardware further contributes to Operating Room (OR) clutter while still requiring manual realignment when patient position changes. Registration challenges are particularly acute in procedures requiring precise visualization and instrument navigation. For example, laparoscopic surgeons face cognitive challenges from the spatial separation between the visual and physical aspects of procedures. The decoupling of visual and motor axes, combined with limited viewing angles, makes accurate depth perception and orientation difficult to maintain throughout complex interventions.

Registration systems enable tracking and alignment of physical objects, medical devices, and imaging data within defined coordinate spaces. These systems typically use various tracking methods to identify and maintain spatial relationships between physical and virtual elements during medical procedures. Current registration systems struggle to effectively register and fuse different imaging modalities like CT, MRI, PET, ultrasound and fluoroscopy. The inability to seamlessly integrate multiple imaging types limits surgeons' ability to leverage complementary visualization approaches. Additionally, existing registration methods often cannot accommodate dynamic changes in patient position or anatomy deformation during procedures. Registration methods can include manual alignment of physical and virtual elements, automated tracking through various sensor types, or hybrid approaches that combine multiple tracking modalities to maintain spatial relationships throughout procedures.

Accordingly, there is a continuing need for improved medical device and imaging registration systems that can maintain accurate alignment throughout procedures without requiring complex external hardware. Desirably, such systems would provide continuous multi-modal tracking capabilities, streamlined setup procedures, and the flexibility to adapt registration approaches based on specific procedural requirements while maintaining compatibility with existing surgical workflows.

### SUMMARY

In concordance with the instant disclosure, improved medical device and imaging registration systems that can maintain accurate alignment throughout procedures without requiring complex external hardware have surprisingly been discovered.

In one embodiment, a method for augmented reality medical guidance can include providing an augmented reality system including a head-mounted display device and providing a tracking system capable of determining position and orientation of tracked objects in a tracking coordinate system. The method can include establishing a first coordinate transformation between the tracking coordinate system and an augmented reality coordinate system of the augmented reality system, thereby achieving device-level registration. The method can further include obtaining medical imaging data of a patient's anatomy, the medical imaging data defining an imaging coordinate system, and establishing a second coordinate transformation between the imaging coordinate system and a patient coordinate system representing the patient's physical anatomy, thereby achieving anatomical registration. A tracking sensor can be attached to a medical instrument, and the position and orientation of the medical instrument can be determined using the tracking system. The position and orientation of the medical instrument can be transformed through the first coordinate transformation to determine a display position in the augmented reality coordinate system. Using the augmented reality system, holographic content derived from the medical imaging data can be displayed, the holographic content positioned using the second coordinate transformation to appear spatially aligned with the patient's physical anatomy. Additionally, a virtual representation of the medical instrument can be displayed at the display position, enabling the physician to view the tracked instrument location relative to the patient's anatomy through the head-mounted display device.

In another embodiment, a method for multi-modal visualization and guidance in an augmented reality medical procedure can include providing an augmented reality system and a tracking system with registration established between a tracking coordinate system, an augmented reality coordinate system, and an imaging coordinate system of pre-procedural medical imaging data. A tracking sensor can be attached to an ultrasound probe, and the ultrasound probe can be positioned on a patient to acquire a real-time ultrasound image. The position and orientation of an ultrasound image plane can be determined using the tracking sensor. Using the augmented reality system, a holographic representation of the ultrasound image can be displayed as a plane positioned in three-dimensional space at the actual location and orientation of the ultrasound image plane, providing ultrasound flashlight visualization. The real-time ultrasound image can be fused with the pre-procedural medical imaging data, enabling simultaneous visualization of real-time and pre-procedural anatomy. Guidance information can be displayed for navigating a tracked interventional device to a target marker, providing the physician with multi-modal imaging guidance that combines the benefits of real-time ultrasound imaging with the comprehensive anatomical context of pre-procedural imaging.

In a further embodiment, a method for dynamically maintaining registration accuracy during an augmented reality medical procedure can include establishing initial registration between a tracking coordinate system, an augmented reality coordinate system, and an imaging coordinate system. Holographic content can be displayed based on the initial registration, the holographic content appearing spatially aligned with a patient's physical anatomy. The system can monitor for conditions affecting registration accuracy during the medical procedure, such as patient motion, respiratory motion, table movement, or anatomical deformation. Upon detecting occurrence of a condition affecting registration accuracy, the system can determine an updated coordinate transformation that compensates for the detected condition. The updated coordinate transformation can be applied to adjust positions of the displayed holographic content to maintain spatial alignment with the patient's physical anatomy despite the changing conditions. This dynamic compensation can ensure that the holographic guidance remains accurately aligned throughout the procedure, even as the patient moves, breathes, or experiences anatomical changes, thereby maintaining the clinical utility of the augmented reality guidance system throughout the entire medical procedure.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is schematic diagram of augmented reality guidance system including augmented reality system, tracking system, and network enabling communication between components.

FIGS. 2A-2B are flowcharts illustrating method for establishing registration between coordinate systems in augmented reality medical guidance system.

FIG. 3 is flowchart illustrating method for multi-modal visualization and guidance in augmented reality medical procedure.

FIG. 4 is flowchart illustrating method for dynamically maintaining registration accuracy during augmented reality medical procedure.

FIG. 5 illustrates holographic content including segmented anatomical structures and MPR slices from multiple imaging sources displayed above a patient body in an augmented reality view.

FIG. 6 illustrates intersecting multi-planar reconstruction (MPR) slices displayed as holographic content in three-dimensional space relative to a patient anatomy.

FIG. 6A illustrates an enlarged view of the intersecting multi-planar reconstruction (MPR) slices shown in FIG. 6.

FIG. 7 illustrates holographic content including segmented anatomical structures, MPR slices aligned with a patient body, a virtual needle representation, and a holographic light ray (HLR) extending from the virtual needle toward a target location.

FIG. 8A illustrates a CT+MRI fusion and segmentation configuration showing pre-procedural CT and MRI imaging data combined through image fusion algorithms with segmented anatomical structures and MPR slices displayed as holographic content with target markers.

FIG. 8B illustrates a CT+MRI fusion configuration showing pre-procedural CT and MRI imaging data combined through image fusion algorithms with MPR slices generated from fused data displayed as holographic content with target markers, without comprehensive anatomical segmentation.

FIG. 8C illustrates a CT+MRI configuration showing pre-procedural CT and MRI imaging data without fusion or segmentation, with MPR slices generated independently from either imaging modality displayed as holographic content with target markers.

FIG. 8D illustrates a CT-only configuration showing pre-procedural CT imaging data without MRI, fusion, or segmentation, with MPR slices generated from CT data displayed as holographic content with target markers.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the terms ‘interventional device’ or ‘tracked instrument’ refers to any medical instrument used during a medical procedure, including but not limited to needles, probes, scalpels, trocars, screwdrivers, forceps, syringes, and other surgical tools and instruments.

As used herein, the term ‘tracking system’ refers to something used to observe one or more objects undergoing motion and supply a timely ordered sequence of tracking data (e.g., location data, orientation data, or the like) in a tracking coordinate system for further processing. As an example, the tracking system can include electromagnetic tracking that can observe an interventional device equipped with a tracker, optical tracking that can detect reflective markers or light sources, fiber optic shape sensing for flexible catheters and instruments within the body, electro-anatomical mapping, impedance-based tracking, or other tracking modalities that can observe the interventional device as it enters, moves through and/or in, and exits a patient's body, as well as while outside of the patient's body.

As used herein, the term “tracking data” refers to information recorded by the tracking system related to an observation of one or more objects undergoing motion.

As used herein, the term “head-mounted device” or “headset” or “HMD” refers to a display device, configured to be worn on the head, that has one or more display optics (including lenses) in front of one or more eyes. These terms may be referred to even more generally by the term “augmented reality system” or “mixed reality system,” where mixed reality refers to interactive environments that combine physical objects with their related data in a manner similar to Medical Internet of Things (MIOT) applications. It should be appreciated that the term “augmented reality system” is not limited to display devices configured to be worn on the head. In some instances, the head-mounted device can also include a non-transitory memory and a processing unit. Examples of suitable head-mounted devices include Microsoft HoloLens® head-mounted device (Microsoft, Redmond, Washington), Magic Leap 2 (Magic Leap, Inc.), DigiLens (DigiLens Inc.) devices, and XREAL Aura (XREAL, Inc.).

As used herein, the terms “imaging system,” “image acquisition apparatus,” “image acquisition system” or the like refer to technology that creates a visual representation of the interior of a patient's body. For example, the imaging system can be a computed tomography (CT) system, a fluoroscopy system, positron emission computed tomography, magnetic resonance imaging (MRI) system, an ultrasound (US) system including contrast agents, color flow doppler, 4D ultrasound with volume rendering capabilities, or the like.

As used herein, references to specific imaging modalities such as ‘CT’ or ‘MRI’ are intended to be exemplary only. Any reference to a particular imaging modality should be understood to encompass CT, MRI, PET, and other tomographic imaging modalities that can be used interchangeably within the system for visualization, registration, and guidance purposes, unless explicitly stated otherwise.

As used herein, the terms “coordinate system” or “augmented reality system coordinate system” or “augmented reality system coordinates” refer to a 3D Cartesian coordinate system that uses one or more numbers to determine the position of points or other geometric elements unique to the particular augmented reality system or image acquisition system to which it pertains. This includes coordinate data from both internal sensors within the headset (such as inertial measurement units and optical sensors) as well as external sensors that may be attached to the headset and/or user for enhanced tracking capabilities. For example, 3D points in the headset coordinate system can be translated, rotated, scaled, or the like, from a standard 3D Cartesian coordinate system.

As used herein, the terms “image data” or “imaging dataset” or “imaging data” refers to information recorded by the imaging system related to an observation of the interior of the patient's body. For example, the “image data” or “imaging dataset” can include processed two-dimensional or three-dimensional images or models such as imaging images, e.g., represented by data formatted according to the Digital Imaging and Communications in Medicine (DICOM) standard or other relevant imaging standards.

As used herein, the terms “imaging coordinate system” or “image acquisition system coordinate system” refers to a coordinate system that uses one or more numbers to determine the position of points or other geometric elements unique to the particular imaging system. This includes three-dimensional orthogonal coordinate systems such as Cartesian coordinates, cylindrical coordinates, spherical coordinates, bipolar cylindrical coordinates, bispherical coordinates, confocal ellipsoidal coordinates, and other orthogonal curvilinear coordinate systems, as well as quaternions for representing three-dimensional rotations. For example, points and vectors in the imaging coordinate system can be translated, rotated, scaled, or the like, to the Augmented Reality system (head mounted displays) coordinate system.

As used herein, the terms “hologram”, “holographic,” “holographic projection”, or “holographic representation” refer to a computer-generated image stereoscopically projected through the lenses of a headset. Generally, a hologram can be generated synthetically (in an augmented reality (AR)) and is not a physical entity.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two or more things being linked or coordinated. For example, a coil-sensor can be integrated with an interventional device. As used herein, the term “real-time” or “near-real time” or “live” refers to the actual time during which a process or event occurs. In other words, a real-time event is done live (within milliseconds so that results are available immediately as feedback). For example, a real-time event can be represented within 100 milliseconds of the event occurring.

As referred to herein, the term “sensor fusion” refers to the process of combining data from multiple sensors or sensor modalities to produce a more accurate, reliable, or complete result than could be achieved using any single sensor or modality alone. In the context of tracking, sensor fusion typically combines data from multiple tracking modalities (e.g., electromagnetic, optical, and inertial) to produce position and orientation estimates that compensate for the limitations of individual modalities.

As referred to herein, the term “registration” refers to the process of determining the spatial relationship between two or more coordinate systems. Once registration is established, positions in one coordinate system can be transformed to corresponding positions in another coordinate system. Registration enables virtual content to be displayed in spatially correct locations relative to real-world objects. Registration may be rigid (preserving distances and angles) or non-rigid (allowing for deformation).

As referred to herein, the term “multi-planar reconstruction” or “MPR” refers to a technique for visualizing medical imaging data as two-dimensional slices in arbitrary orientations. While medical images are typically acquired in standard planes (axial, sagittal, coronal), multi-planar reconstruction allows visualization of slices in any orientation, including oblique planes aligned with anatomical structures or interventional devices.

As referred to herein, the term “segmentation” refers to the process of identifying and extracting specific structures from medical images. Segmentation may be manual (user-delineated), semi-automatic (user-guided with automatic processing), or fully automatic (algorithm-based without user intervention). Segmentation produces a three-dimensional representation of anatomical structures such as organs, bones, vessels, or pathological structures such as tumors. The segmented structures can be visualized as holographic overlays.

As referred to herein, the term “fiducial” or “fiducial marker” refers to a marker placed on or attached to a patient to provide a reference point visible in both medical images and the real world. Fiducials may be radiopaque (visible in X-ray, CT, or fluoroscopy), MRI-visible, visible to optical sensors, or detectable by multiple modalities. Fiducials enable registration by providing corresponding points that can be identified in imaging data and in the physical world.

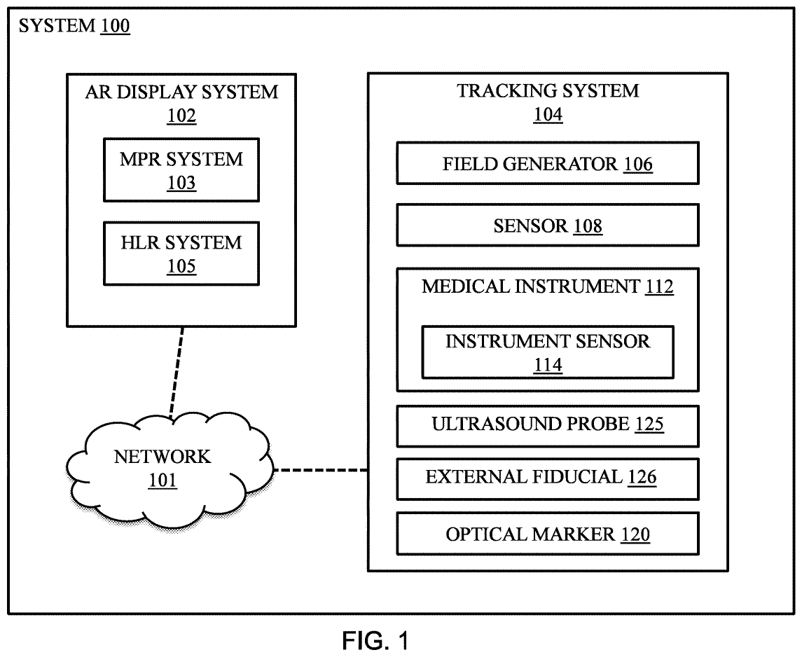

FIG. 1 illustrates a high-level architecture of an augmented reality guidance system 100 according to one embodiment. The system 100 can include an augmented reality system 102, a tracking system 104, and a network 101 enabling communication between components. The augmented reality system 102 can provide visualization of holographic content overlaid on the user's view of the real world. The tracking system 104 can determine position and orientation of tracked objects including the augmented reality system 102 itself and medical instruments. The network 101 can enable data communication between system components.

The augmented reality system 102 can present holographic content at specific three-dimensional locations such that the content appears positioned at particular locations in the real world. The holographic content can be displayed within the surgical bed, above the patient body, or at any location in the surrounding environment as viewed through holographic lenses or headsets worn by the physician or surgical team. For example, a virtual representation of a tumor extracted from medical imaging can be displayed at the correct anatomical location inside the patient body, appearing to float within the surgical bed when viewed through the head-mounted display device. A virtual representation of a tracked needle or other surgical instrument can indicate the current position of the actual physical instrument during tracking. The system can maintain spatial tracking of the display device position and orientation, either through built-in sensors or through the tracking system 104, enabling holographic content to maintain correct spatial relationships with real-world objects as the user moves.

In specific embodiments, the augmented reality system 102 can include a display device capable of presenting virtual content overlaid on or integrated with a view of the real-world environment. The display device can be a head-mounted display (HMD) device worn by a physician or medical professional during a procedure, a handheld display device such as a tablet or smartphone, a projector-based system projecting virtual content onto physical surfaces, or other display technologies capable of augmented reality visualization. Head-mounted displays can provide hands-free operation while the display moves naturally with head movements. The head-mounted display can use optical see-through technology (virtual content displayed on transparent or semi-transparent optics through which the user directly views the real world) or video see-through technology (cameras capture real-world video displayed to the user with virtual content composited into the video stream). Optical see-through can provide little to no latency in the real-world view and no dependence on camera quality. Video see-through can provide precise alignment between real-world video and virtual content and can enable image processing of the real-world view. Suitable head-mounted display devices can include Microsoft HoloLens (first and second generation), Magic Leap One and Magic Leap 2, Meta Quest Pro, Varjo XR-3, and other augmented reality or mixed reality headsets. Selection factors can include field of view, display resolution, display brightness, tracking accuracy, processing power, comfort, battery life, and cost.

The head-mounted display device can include built-in sensors for tracking its own position and orientation. These sensors can typically include cameras for visual environment tracking (inside-out tracking), inertial measurement units for head motion tracking, and optionally depth sensors for three-dimensional environment geometry. The device can use these sensors to track its position and orientation within the room (simultaneous localization and mapping or “SLAM”), enabling display of holographic content at fixed locations in room coordinates that appear stationary as the user moves.

The augmented reality system 102 can include processing hardware for executing application software. Processing hardware can include central processing units (CPUs), graphics processing units (GPUs), digital signal processors (DSPs), application-specific integrated circuits (ASICs), field-programmable gate arrays (FPGAs), or other processing devices. Processing can occur on processors within the head-mounted display device itself, on external computing devices (desktop computers, laptop computers, mobile devices, or servers), or distributed across multiple devices communicating via the network 101.

A multi-planar reconstruction (MPR) system 103 can process three-dimensional medical imaging data (CT, MRI, and/or ultrasound datasets) to generate two-dimensional slice views. The MPR system 103 can generate slices in standard orientations (axial, sagittal, coronal) or arbitrary oblique orientations. The MPR system 103 can generate “peri-axial” and “peri-sagittal” slices positioned to bisect a tracked interventional device such as a needle, providing views along and perpendicular to the device's trajectory. The MPR system 103 can apply windowing (adjusting brightness and contrast to optimize visualization of tissue types), edge detection, thresholding, or other image processing operations.

A holographic light ray (HLR) system 105 can generate guidance visualizations assisting navigation of interventional devices, i.e. medical instruments, to target locations. The HLR system 105 can display a virtual line or ray extending from the current position of a tracked instrument toward a target location, projecting the instrument's trajectory. The HLR can be color-coded to indicate proximity to target, risk of intersection with critical structures, or other relevant information. The HLR system 105 can update the holographic light ray in real-time as the tracked instrument moves.

The augmented reality system 102 can display various holographic content types including: three-dimensional surface meshes representing segmented anatomical structures; two-dimensional images positioned in three-dimensional space (MPR slices); lines and rays (HLR projections); geometric markers (target spheres or ellipsoids); text labels; and three-dimensional models of equipment (virtual representations of the electromagnetic field generator or tracked instruments). Visual appearance can be controlled through color, transparency, brightness, and rendering style (solid, wireframe, or point cloud).

The augmented reality system 102 can include built-in sensors utilized for various purposes. Most head-mounted display devices can include cameras for environment tracking that can also be used for optical tracking of instruments, registration devices, or fiducial markers. Many can include depth sensors (structured light, time-of-flight, or stereo cameras) generating three-dimensional environment geometry, usable for surface scanning to assist registration or detecting patient motion. Inertial measurement units can provide supplementary tracking information for sensor fusion.

The network 101 can provide communication pathways between the augmented reality system 102, the tracking system 104, and other system components (medical imaging systems, ultrasound systems, hospital information systems, or external computing devices). The network 101 can provide data communication with sufficient bandwidth and sufficiently low latency to support real-time operation. For tracking data, real-time operation can typically require update rates of at least 20 Hz, preferably 30-60 Hz or higher.

The network 101 can include wired connections (Ethernet, USB, or other wired protocols) or wireless connections (Wi-Fi, Bluetooth, or proprietary wireless protocols), or combinations thereof. Wired connections can provide high bandwidth, low latency, and high reliability but may constrain physical movement if the head-mounted display device is tethered. Wireless connections can provide greater freedom of movement but may have bandwidth, latency, or reliability limitations depending on protocol and environmental factors such as interference. In embodiments where processing is distributed across multiple computing devices, the network 101 can include local area networks (LANs) connecting devices within a procedure room, wide area networks (WANs) connecting devices at different locations, or cloud-based communication through internet-connected servers. Network architecture selection can depend on bandwidth requirements, latency requirements, security considerations, reliability requirements, and integration with existing hospital information technology infrastructure.

In some embodiments, processing tasks can be performed locally within the augmented reality system 102 itself, reducing network traffic and eliminating network latency for those tasks. For example, holographic content rendering can be typically performed by the graphics processing unit within the head-mounted display device. As processing capabilities of head-mounted display devices improve, more processing tasks can be performed locally, reducing dependence on external computing devices and network communication.

The tracking system 104 can provide real-time position and orientation information for one or more objects in three-dimensional space. The tracking system 104 can generally include one or more sensors associated with tracked objects and a mechanism for processing sensor data to determine position and orientation of the tracked objects. The tracking system 104 can operate continuously during a procedure, providing updated position and orientation data at regular intervals.

The tracking system 104 can determine position and orientation of tracked objects within a tracking coordinate system serving as a reference frame. Position can be specified as three translational coordinates (x, y, z). Orientation can be specified as three rotational coordinates (roll, pitch, yaw). A tracking system providing position and orientation can provide six degrees of freedom (6-DOF) tracking. A tracking system providing only position can provide three degrees of freedom (3-DOF) tracking. Intermediate values (4-DOF, 5-DOF) can provide position and partial orientation information.

The tracking system 104 can employ a single tracking modality or multiple tracking modalities. A single-modality tracking system can use one type of tracking technology, such as electromagnetic tracking. A multi-modal tracking system can use multiple types of tracking technology, such as electromagnetic tracking combined with optical tracking and inertial tracking. Multi-modal tracking systems can provide improved accuracy, improved reliability, larger working volume, and compensation for limitations inherent in individual modalities.

Electromagnetic tracking can determine position and orientation of sensors by measuring electromagnetic fields. An electromagnetic tracking system can include at least one electromagnetic field generator and at least one electromagnetic sensor. The field generator can produce time-varying electromagnetic fields, typically using multiple coils oriented in different directions. The sensor can contain one or more coils that detect the electromagnetic fields. By analyzing the sensor signals, the tracking system can compute position and orientation of each sensor relative to the field generator. Electromagnetic tracking can offer the advantage of not requiring line-of-sight between sensors and the field generator, allowing sensors to be tracked even when occluded by the patient's body or surgical drapes. However, electromagnetic tracking can be affected by metallic objects in the tracking volume, which can distort the electromagnetic field and degrade accuracy.

The electromagnetic field generator 106 can produce electromagnetic fields enabling position and orientation determination for electromagnetic sensors. The field generator can include multiple electromagnetic coils, drive electronics supplying time-varying currents to the coils, and typically a housing positioning and protecting the coils. The field generator 106 can be configured in various form factors. Planar electromagnetic field generators can have coils arranged in a substantially flat configuration, suitable for placement on a flat surface such as a procedure table, positioned beneath or adjacent to the patient. Tabletop implementations can be positioned on the surface of the procedure table, typically beneath a patient support pad. Articulating arm-mounted implementations can attach the field generator 106 to a flexible or rigid positioning arm allowing positioning with multiple degrees of freedom. Window implementations can integrate the field generator 106 into or adjacent to a flat panel that can be positioned relative to the patient. Wireless field generator configurations can eliminate the need for a power cable by including an internal battery. Specific commercial examples can include the NDI Aurora planar field generator, the NDI Aurora compact field generator, Ascension Technology Corporation field generators including the planar transmitter and mid-range transmitter, and Polhemus field generators.

Electromagnetic sensors 108 can detect electromagnetic fields generated by the field generator 106 and produce signals enabling determination of sensor position and orientation. An electromagnetic sensor can include one or more coils that generate electrical signals in response to time-varying electromagnetic fields.

A 5-DOF sensor can include coils enabling determination of three position coordinates and two orientation coordinates (typically azimuth and elevation). A 6-DOF sensor can include coils enabling determination of three position coordinates and three orientation coordinates (full orientation including rotation about the sensor's longitudinal axis). Typical sensor sizes for medical applications can range from approximately 0.5 mm diameter to approximately 8-10 mm diameter. Electromagnetic sensors can be wired or wireless. Wired sensors can include a cable carrying sensor signals from the sensor to tracking system electronics. Wireless sensors can include electronics within the sensor for signal processing and wireless communication (such as Bluetooth, WiFi, or proprietary wireless protocols) and can include a power source such as a battery. One exemplary wireless sensor is the AMFITRACK sensor, which can combine an electromagnetic sensor with an inertial measurement unit (IMU) and magnetometer in a wireless package. The AMFITRACK sensor can communicate tracking data wirelessly, typically using Bluetooth Low Energy (BLE). The sensor can include a rechargeable battery providing several hours of continuous operation. The AMFITRACK sensor can provide 6-DOF electromagnetic tracking plus inertial data from the IMU and magnetometer data, enabling multi-modal sensor fusion.

Electromagnetic sensors can be mounted to tracked objects, for example medical instrument 112 using various attachment methods. Adhesive mounting can use medical-grade adhesive to attach the sensor directly to the object surface. Mechanical mounting can use clips, brackets, or housings that grip the instrument mechanically. Embedded mounting can integrate the sensor within the instrument structure itself. For ultrasound probe 125 tracking, brackets can attach electromagnetic sensors to ultrasound probes without interfering with probe manipulation or image quality. Specific commercial electromagnetic sensors can include NDI Aurora 6-DOF sensors (model numbers 610028, 610054, and others), which can be compact cylindrical sensors approximately 0.5 mm to 1.3 mm in diameter. Ascension Technology Corporation can provide both wired and wireless electromagnetic sensors including the trakSTAR sensors.

Optical tracking can use cameras or other optical sensors to determine position and orientation based on visual information. Optical tracking can involve capturing images of tracked objects or markers, processing the images to identify and localize the objects or markers within the image, and computing three-dimensional position and orientation from the two-dimensional image information. Optical tracking systems can be categorized as active or passive. Active optical tracking can use markers that emit light, typically infrared light-emitting diodes (LEDs). Passive optical tracking can use markers that reflect light, such as retroreflective spheres or surface markers. Optical tracking can have the fundamental requirement of line-of-sight visibility: the markers must be visible to the camera or cameras.

Optical tracking technologies can include image target detection (using two-dimensional printed patterns recognized by computer vision algorithms), model target detection (using three-dimensional models of physical objects), optical code recognition (using fiducial markers such as bar codes, QR codes, ArUco markers, or AprilTags), active infrared tracking (using infrared LEDs as markers), passive infrared tracking (using retroreflective markers), depth sensing (using structured light, time-of-flight, or stereoscopic methods), headset-mounted camera tracking, and external camera-based tracking.

Image target detection can use printed two-dimensional patterns recognized by computer vision algorithms. Software libraries such as Vuforia can provide image target detection capabilities. In the present system, image targets can be attached to the electromagnetic field generator 106 to enable optical tracking of the field generator position. The electromagnetic field generator 106 is typically positioned on or near the surgical table and remains stationary during the procedure, creating a stable electromagnetic tracking volume. The optical tracking of the field generator position provides device-level registration between the augmented reality coordinate system and the tracking coordinate system, offering an alternative to manual alignment of the generator hologram or to attaching an electromagnetic sensor to the augmented reality headset. The optical tracking system in this implementation tracks the field generator for registration purposes, while the electromagnetic sensors attached to medical instruments are tracked by the electromagnetic tracking system 104 itself, not by optical tracking.

Model target detection can use three-dimensional models of physical objects and recognize the objects in camera images by matching the observed appearance to the expected appearance based on the three-dimensional model. Software libraries such as Vuforia can provide model target detection capabilities.

Active infrared LED tracking can use LEDs that emit infrared light at wavelengths typically between 850 nm and 950 nm. Each tracked object can include multiple LEDs arranged in an asymmetric pattern. In one embodiment, active LED tracking can be used to track the electromagnetic field generator 106 by attaching a rigid body with multiple LEDs to the field generator, enabling automatic registration between the electromagnetic tracking coordinate system and the head-mounted display coordinate system.

Passive infrared tracking can use retroreflective markers, typically spherical markers ranging from approximately 5 mm to 20 mm in diameter. Infrared illuminators positioned near cameras can illuminate the scene, and the retroreflective markers can appear as bright spots in infrared camera images. Multiple markers can be arranged in asymmetric patterns on rigid bodies.

Depth sensing cameras can provide three-dimensional geometry information usable for surface scanning and tracking. The Microsoft HoloLens 2 can include a depth sensor based on time-of-flight technology. Other depth sensors can include the Intel RealSense family, the Microsoft Azure Kinect, and various LiDAR sensors. In the present system, depth sensing can be used to capture the three-dimensional geometry of the patient's skin surface, which can be compared to a skin surface extracted from pre-procedural imaging to establish anatomical registration.

Headset-mounted camera tracking can use cameras integrated into the augmented reality headset. Most augmented reality headsets can include multiple cameras for environment tracking that can also be used for tracking markers, objects, or surfaces. External camera-based tracking can use cameras positioned in fixed locations in the room or on mobile stands, providing a consistent viewpoint regardless of where the user looks.

Stylus-based optical tracking can use a tracked stylus as a pointing device for registration. The stylus can be a rigid body with markers (LEDs or retroreflective spheres) and a sharp tip. The physician can use the stylus to point to locations on the patient's body (such as anatomical landmarks or fiducial markers), and the tracking system can record the three-dimensional position of each point.

Inertial measurement units (IMUs) can measure motion using accelerometers and gyroscopes. Accelerometers can measure linear acceleration along one or more axes. Gyroscopes can measure angular velocity (rotational rate) about one or more axes. By integrating acceleration over time, position changes can be computed. By integrating angular velocity over time, orientation changes can be computed.

IMUs can provide several advantages including very high update rates (typically 100 Hz to 1000 Hz or higher), self-contained operation without requiring external infrastructure, immunity to metallic interference or line-of-sight occlusion, and small, lightweight, low cost construction. However, IMUs can have a fundamental limitation: drift. Small errors in acceleration and angular velocity measurements can accumulate over time when integrated to determine position and orientation. Due to drift, IMUs can be used in combination with other tracking modalities through sensor fusion. The IMU can provide high-frequency motion information with low latency, while electromagnetic tracking or optical tracking can provide absolute position and orientation information that corrects for IMU drift. Sensor fusion algorithms (such as Kalman filters) can optimally combine the complementary information from multiple sensors. A basic IMU can include a three-axis accelerometer and a three-axis gyroscope, providing six sensor signals. Some IMUs can also include a three-axis magnetometer, which can measure the Earth's magnetic field, providing orientation information relative to magnetic north. An IMU with accelerometer, gyroscope, and magnetometer can be called a 9-axis IMU. Specific examples of IMU chips can include the Bosch BMI088 (6-axis IMU), the STMicroelectronics LSM6DSO (6-axis IMU), the InvenSense ICM-20948 (9-axis IMU), and others from manufacturers including Analog Devices, NXP, and others.

Additional tracking modalities can include acoustic tracking, fiber optic shape sensing, electro-anatomical mapping, impedance-based tracking, and various radio-frequency-based tracking technologies. Each modality can have specific characteristics making it suitable for particular applications.

Acoustic tracking can use ultrasonic sound waves to determine position. Transmitters (such as piezoelectric transducers) can emit ultrasonic pulses, and receivers can detect the pulses. Time-of-flight of sound waves can determine distance, and triangulation can determine three-dimensional position. The Sensoryx tracking system can combine acoustic tracking with optical tracking and inertial tracking in a multi-modal sensor fusion system.

Fiber optic shape sensing can use fiber optic cables with embedded Fiber Bragg Gratings (FBGs) or Rayleigh scattering to measure the shape of flexible instruments such as catheters or needles. By analyzing light transmitted through or reflected from multiple points along the fiber, the three-dimensional shape of the fiber can be reconstructed.

Radio-frequency (RF) based tracking technologies can include RFID (Radio Frequency Identification), Bluetooth Low Energy (BLE), WiFi, and Ultra-Wideband (UWB). RFID can use passive or active tags that respond to interrogation signals. BLE and WiFi can use signal strength measurements to estimate distance. UWB can use time-of-flight measurements with very short pulses, providing positioning accuracy on the order of 10-30 cm.

Hybrid or multi-modal tracking systems can combine two or more tracking modalities to achieve tracking performance exceeding what any single modality can provide. Different tracking modalities can have complementary strengths and limitations. By combining multiple modalities and intelligently processing the combined data, a hybrid system can leverage the strengths of each modality while compensating for limitations. Multi-modal tracking can provide improved accuracy by combining measurements from multiple sources, improved reliability by providing redundancy, extended working volume or operating conditions by using different modalities in different regions or situations, and improved update rate and reduced latency by using high-frequency modalities to interpolate between lower-frequency modalities.

Electromagnetic and optical hybrid systems can combine the advantages of electromagnetic tracking (no line-of-sight requirement) with optical tracking (high accuracy, no metallic interference). Electromagnetic+inertial hybrid systems can combine the absolute position and orientation from electromagnetic tracking with the high-frequency motion data from inertial tracking. Optical+inertial hybrid systems can combine visual tracking with IMU for high-frequency motion updates.

The AMFITRACK wireless sensor can exemplify an electromagnetic+inertial hybrid system. The sensor housing can contain a 6-DOF electromagnetic sensor coil, a 9-axis IMU (with accelerometer, gyroscope, and magnetometer), wireless communication electronics (Bluetooth Low Energy), and a rechargeable lithium-ion battery. The sensor firmware can implement sensor fusion algorithms that combine electromagnetic tracking and inertial tracking data. The fused tracking output can be transmitted wirelessly at update rates of 100 Hz or higher. The sensor fusion algorithm can use an Extended Kalman Filter that maintains a state estimate including position, velocity, orientation, and angular velocity. The IMU measurements can provide prediction steps updating the state estimate based on measured motion. The electromagnetic tracking measurements can provide correction steps adjusting the state estimate based on absolute position and orientation. The algorithm can adaptively weight contributions from each modality based on estimated quality.

For hybrid systems combining electromagnetic and optical tracking, automatic modality switching can occur based on line-of-sight visibility and electromagnetic quality metrics. If optical markers are visible and electromagnetic quality is degraded, the system can switch to optical tracking. If markers become occluded but electromagnetic quality is good, the system can switch to electromagnetic tracking.

Registration devices can optionally be used to facilitate establishment of coordinate transformations between different coordinate systems. A registration device can be a physical object that can be tracked by the tracking system 104 and localized in the real world or medical imaging data. Registration devices are not required for system operation; alternative registration methods described in subsequent sections can achieve registration without additional hardware. When used, registration devices can include one or more tracking sensors enabling position and orientation determination. Registration devices can include features enabling localization through various means, such as fiducial markers visible in medical imaging, optical patterns recognizable by cameras, or distinctive geometry enabling surface-based registration.

Registration devices can serve different purposes in the registration workflow. Some registration devices can combine fiducial markers with tracking sensors for image-based localization and real-time tracking. Other registration devices can be positioned on or relative to the patient's anatomy to serve as a stable reference frame for tracking patient position. Simplified registration devices can be designed specifically for device-level registration (aligning the tracking system with the augmented reality system). Registration devices can be implemented with various sensor types (electromagnetic, optical, inertial, or combinations), various mounting methods (adhesive, clip-on, arm-mounted, table-mounted), and various configurations (single-sensor or multi-sensor, wired or wireless, disposable or reusable). The specific configuration can be selected based on procedural requirements and equipment availability.

The disclosed system can support registration without requiring separate registration devices. Device-level registration can be achieved through automated methods (such as attaching a sensor to the augmented reality headset), manual alignment methods (such as generator hologram alignment), or optical tracking of the field generator 106. Anatomical registration can be achieved through markerless methods such as skin surface alignment, ultrasound reference plane matching, or anatomical landmark-based registration. These alternative approaches are described in detail in subsequent sections.

One or more medical instruments 112 used in image-guided procedures can be equipped with tracking sensors enabling the tracking system 104 to determine the instrument position and orientation in real-time. A tracked medical instrument 112 can include an interventional device (such as a needle, a trocar, an ultrasound probe, a laparoscopic instrument, a catheter, an ablation probe, or a surgical tool) and one or more tracking instrument sensors 114 (referred to herein as tracking sensor) attached to or integrated into the device. The tracking sensor 114 can be attached to the instrument using various attachment mechanisms including brackets, housings, clips, adhesive mounts, or embedded integration. The attachment mechanism can maintain a fixed spatial relationship between the sensor and the clinically relevant portion of the instrument (such as the needle tip, ultrasound image plane, or instrument tip). For instruments 112 entering the sterile field, the tracking sensor 114 and attachment mechanism can be sterilizable, covered with sterile draping, or designed for single-use disposal. Alternatively, metal material surgical instruments can be registered and tracked by detecting distortions in the electromagnetic field caused by the conductive metal material, reducing the need for attached tracking sensors in certain implementations.

The spatial relationship between the tracking sensor 114 and the medical instrument 112 can be determined through calibration. Calibration can establish the offset and orientation from the sensor coordinate system to the instrument's functional coordinate system. Once calibrated, the tracking system 104 can report the position and orientation of the instrument's functional portion by applying the calibration transformation to the tracking sensor 114 position. Calibration parameters can be stored and associated with specific instrument and sensor combinations using device identifiers.

In one particular example, the medical instrument 112 can be an ultrasound probe 125 that can be tracked using brackets or housings that attach tracking sensor 114 to the probe. The bracket can mount to the probe handle or body and position the tracking sensor at a known location and orientation relative to the ultrasound image plane. Brackets can be designed for specific probe types (curvilinear, linear, phased array) or can be adjustable to accommodate multiple probe types. The bracket can include mechanical features such as snap-fit mechanisms for quick attachment, keyed grooves ensuring correct orientation, and backstop features providing repeatable positioning. The bracket can be reusable and sterilizable, or can be designed for single-use applications. The sensor can be integrated into the bracket or can be removable from the bracket. Laparoscopic instruments including laparoscopic ultrasound probes, endoscopes, borescopes, fiberscopes, and camera systems can also be tracked using sensors 114 attached to the proximal end (handle) outside the patient's body. The sensor position combined with the known instrument geometry can enable computation of the distal tip position and orientation. Laparoscopic ultrasound probes can use brackets similar to external ultrasound probes, adapted for the elongated instrument geometry. Tracked laparoscopic camera information can be used to align augmented reality overlays with the laparoscopic video view, enabling surgeons to view both laparoscopic video and augmented reality guidance information simultaneously. This can improve spatial orientation during minimally invasive procedures.

Needles and surgical tools can be tracked using sensors attached to the instrument handle, hub, or shaft. Electromagnetic sensors can be attached to needles using clips or brackets that mount to the needle hub or syringe barrel, positioning the sensor at a known offset from the needle tip. LED marker configurations can be used for optical tracking, with multiple LEDs arranged in an asymmetric pattern on a rigid body attached to the instrument.

Once tracked, a virtual representation 116 of the instrument 112 can be displayed in the augmented reality system 102. The virtual representation 116 can be rendered at the actual position and orientation of the physical tool 112, providing visual feedback about tool position even when the physical tool is partially or fully obscured by the patient's body. The virtual representation 116 can be updated in real-time as the physical tool moves.

The augmented reality guidance system 100 can store imaging data and tracking data generated during a medical procedure for post-procedural review, analysis, or replay. The system 100 can record, in a non-transitory memory, a time-synchronized log of tracking data from the tracking system 104, including the position and orientation of each tracked medical instrument 112 and tracking sensor 114 throughout the procedure. The system 100 can concurrently store the medical imaging data obtained in step 208, holographic content 118 displayed during the procedure, and any real-time imaging data acquired during the procedure, such as ultrasound image planes 128. Coordinate transformations established during registration, including the first and second coordinate transformations established in steps 206 and 210 of method 200, can also be stored to preserve the spatial relationships between coordinate systems as they existed during the procedure. The stored data can be retrieved and replayed to reconstruct the augmented reality guidance view at any point during the procedure, displaying the virtual representation 116 of the medical instrument 112 and holographic content 118 as they appeared in real-time. The stored data can be used for post-procedural review of instrument trajectory and target accuracy, quality assurance, physician training, or retrospective clinical analysis. The non-transitory memory can reside within the augmented reality system 102, on an external computing device communicating via the network 101, or on a remote server or cloud-based storage system. Access to stored procedure data can be controlled through authentication mechanisms to maintain patient data privacy and comply with applicable healthcare data security requirements.

The following sections describe methods for implementing augmented reality medical guidance using the systems, components, and techniques described above. The methods can be practiced using various combinations of hardware components, tracking modalities, and registration approaches, providing flexibility to adapt to different procedural requirements and clinical workflows.

The method 200 describes a multi-stage registration method establishing coordinate transformations between the tracking system 104, the augmented reality system 102, and the patient's anatomy. This method enables tracked medical instruments to be visualized with holographic guidance overlays spatially aligned with the patient's anatomy. Method 200 includes multiple approaches for device-level registration (manual, automated, optical, or stylus-based) and anatomical registration (fiducial-based, surface-based, ultrasound-based, or landmark-based). Method 300 describes a multi-modal visualization and guidance method that fuses real-time ultrasound imaging with pre-procedural or intra-procedural imaging and provides comprehensive guidance for navigating interventional devices to targets. This method enables viewing complementary information from multiple imaging modalities simultaneously with real-time trajectory guidance. Method 400 describes a dynamic registration maintenance method that detects and compensates for changing conditions including patient motion, respiratory motion, table movement, and anatomical deformation. This method maintains registration accuracy throughout procedures despite factors that would otherwise degrade alignment. The methods can be practiced independently or in combination. Each method includes variations and optional steps enabling customization to specific clinical applications.

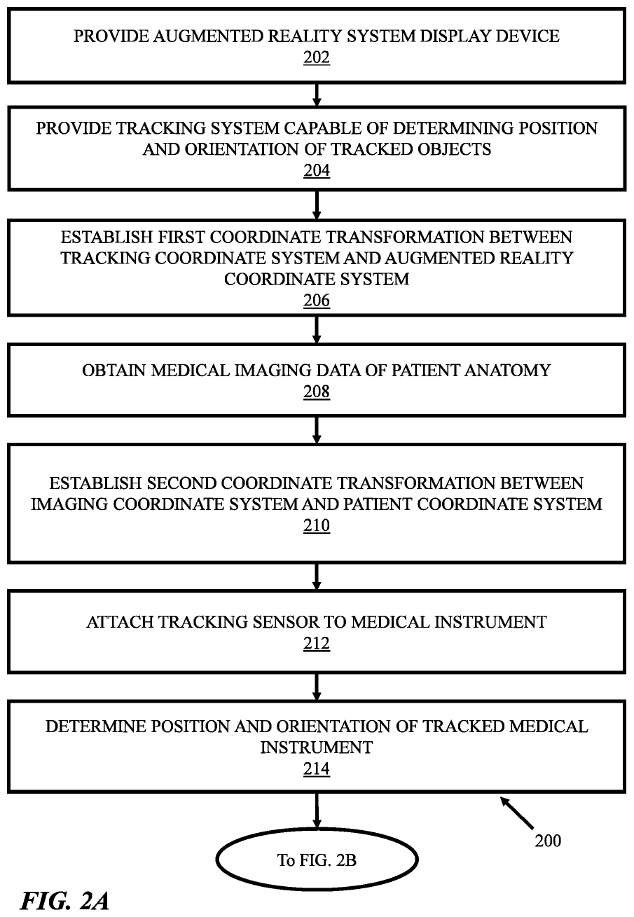

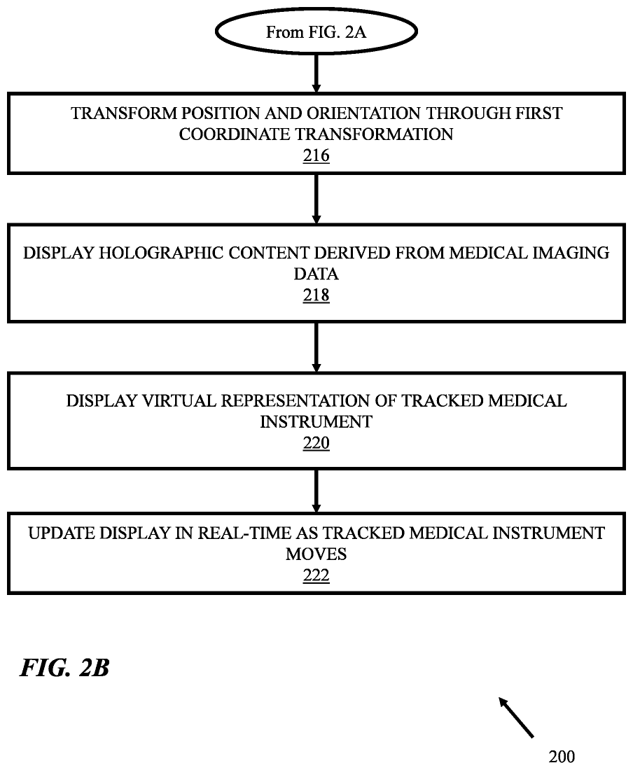

FIGS. 2A-2B illustrates a method 200 for establishing registration between coordinate systems in an augmented reality medical guidance system 100. The method 200 can enable tracked medical instruments 112 to be visualized with holographic guidance overlays that appear spatially aligned with a patient's anatomy. The method 200 can include a step 202 of providing an augmented reality system 102 including a head-mounted display device capable of displaying holographic content 118 overlaid on a user's view of a real-world environment. The head-mounted display device can be worn by a physician or medical professional during a medical procedure. The augmented reality system 102 can be implemented as described hereinabove.

The method 200 can include a step 204 of providing a tracking system 104 capable of determining position and orientation of one or more tracked objects in a tracking coordinate system. The tracking system 104 can use electromagnetic tracking, optical tracking, inertial tracking, acoustic tracking, or combinations thereof as described hereinabove. The tracking system 104 can provide real-time position and orientation updates at rates of 20 Hz or higher, preferably 30-60 Hz or higher. The method 200 can include a step 206 of establishing a first coordinate transformation between the tracking coordinate system and an augmented reality coordinate system of the augmented reality system 102. This first transformation can enable device-level registration, aligning the tracking system 104 with the augmented reality system 102.

In some embodiments, the first coordinate transformation can be established using a manual registration approach. A virtual holographic representation of a component of the tracking system 104 (such as an electromagnetic field generator 106) can be displayed in the augmented reality system 102. The electromagnetic field generator 106 serves as a fixed reference point defining the tracking coordinate system, making it suitable for establishing device-level registration between the tracking system 104 and the augmented reality system 102. The user can manipulate the position and orientation of the virtual holographic representation using hand gestures, voice commands, or controller input until the virtual representation visually aligns with the physical component as viewed through the head-mounted display device. The augmented reality system 102 can display visual feedback indicating alignment quality, such as color-coded indicators, distance metrics, or alignment guides. Upon receiving user confirmation, the augmented reality guidance system 100 can compute the first coordinate transformation based on the confirmed position and orientation of the virtual holographic representation.

Alternatively, the first coordinate transformation can be established using an automated registration approach. One of the tracking sensors 114 can be attached to the augmented reality system 102, such as an electromagnetic sensor mounted to the head-mounted display device. The tracking system 104 can intermittingly or continuously determine the sensor position in the tracking coordinate system, while the augmented reality system 102 can know the sensor position in the augmented reality coordinate system based on the known mounting location. The system 100 can automatically compute the first coordinate transformation based on simultaneous position measurements from both coordinate systems. This automated approach can provide continuous registration that automatically updates if coordinate systems drift over time.

Alternatively, the first coordinate transformation can be established using an optical registration approach. Optical markers 120 (such as infrared LEDs, retroreflective spheres, image targets, or model targets) can be attached to a component of the tracking system 104. Cameras integrated into the augmented reality system 102 can detect and track the optical markers 120, determining the component position in the augmented reality coordinate system. The system 100 can compute the first coordinate transformation from the known position in both coordinate systems.

Alternatively, the first coordinate transformation can be established using a stylus-based registration approach. A tracked stylus can be used to touch multiple known locations on a component of the tracking system 104. The system 100 can record the three-dimensional position of the stylus tip in the tracking coordinate system. The user can also identify the same physical locations in the augmented reality display. The system 100 can compute the first coordinate transformation using point-set registration algorithms that find the optimal transformation aligning the corresponding point sets.

The method 200 can include a step 208 of obtaining medical imaging data of a patient's anatomy. The medical imaging data can be pre-procedural imaging acquired before the procedure begins or intra-procedural imaging acquired during the procedure. Pre-procedural imaging can include computed tomography (CT), multi-detector CT (MDCT), magnetic resonance imaging (MRI), positron emission tomography (PET), ultrasound, or other imaging modalities, or combinations thereof, acquired prior to the procedure and transferred to the augmented reality guidance system 100 for use during guidance. Intra-procedural imaging can include cone beam CT (CBCT) acquired using a C-arm or other interventional imaging system during the procedure, providing updated anatomical information reflecting the patient's current intra-procedural anatomy. The intra-procedural imaging workflow can follow the same processing pipeline as pre-procedural imaging: CBCT data can be loaded into the system 100, processed to generate MPR slices 124, and used to display holographic content 118 aligned with the patient's anatomy. The system 100 can support simultaneous use of pre-procedural and intra-procedural imaging, enabling direct comparison of the two datasets. For example, edge map fusion can display contours and edges extracted independently from MDCT and CBCT data, color-coded to distinguish between the two sources, enabling the physician to assess anatomical correspondence and identify procedurally-induced changes between pre-procedural and intra-procedural imaging. In all cases, the medical imaging data can be three-dimensional volumetric imaging stored in DICOM format defining an imaging coordinate system. The step 208 can include segmenting anatomical structures from the medical imaging data as described hereinabove. Segmented structures 122 can be represented as three-dimensional surface meshes and can be used for anatomical registration and holographic visualization.

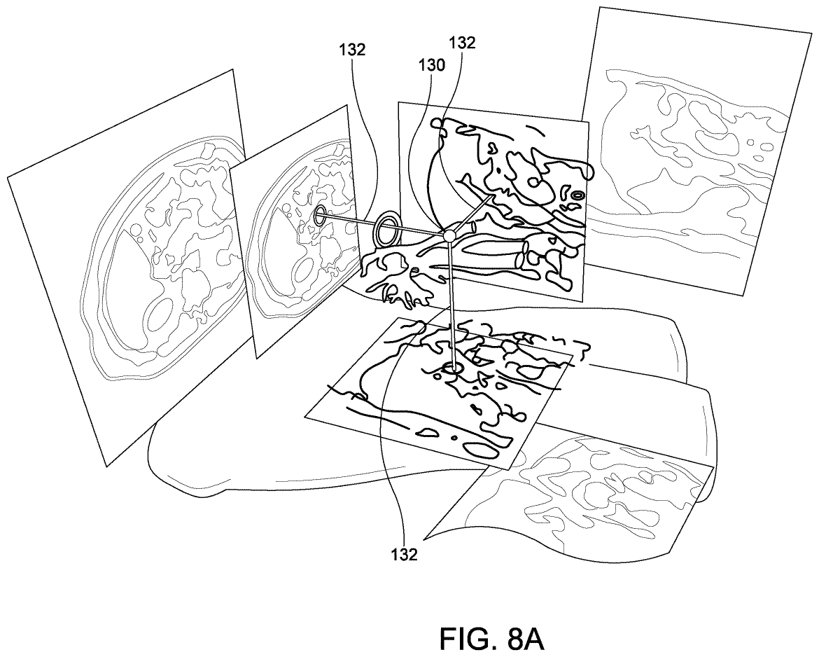

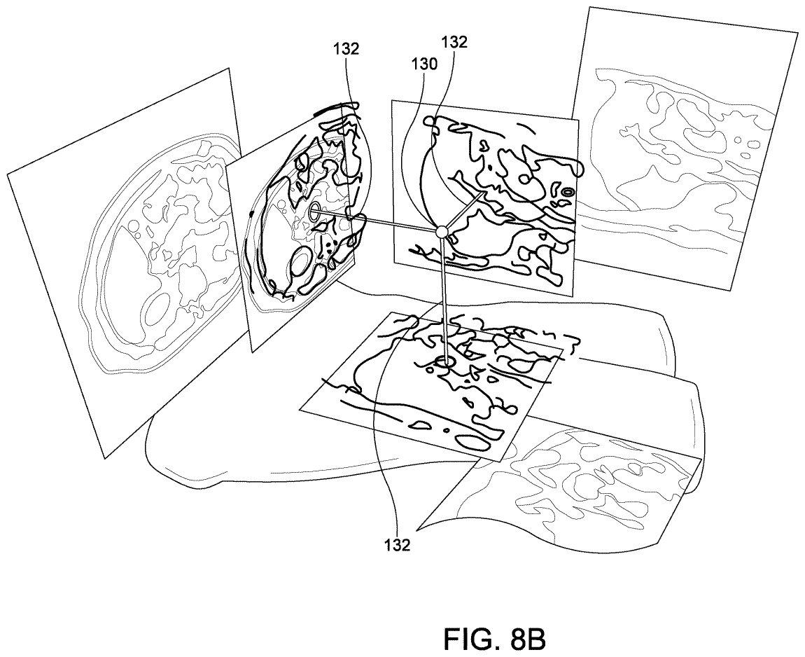

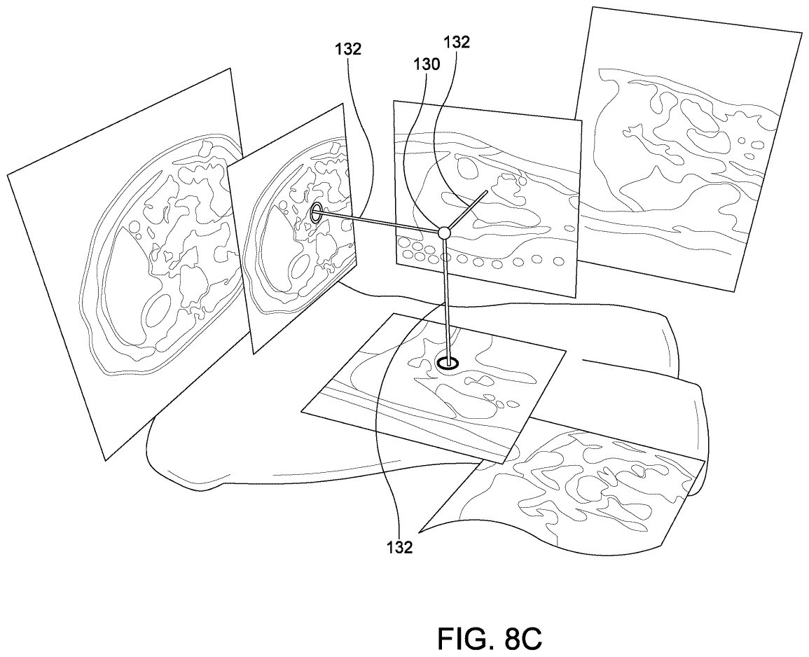

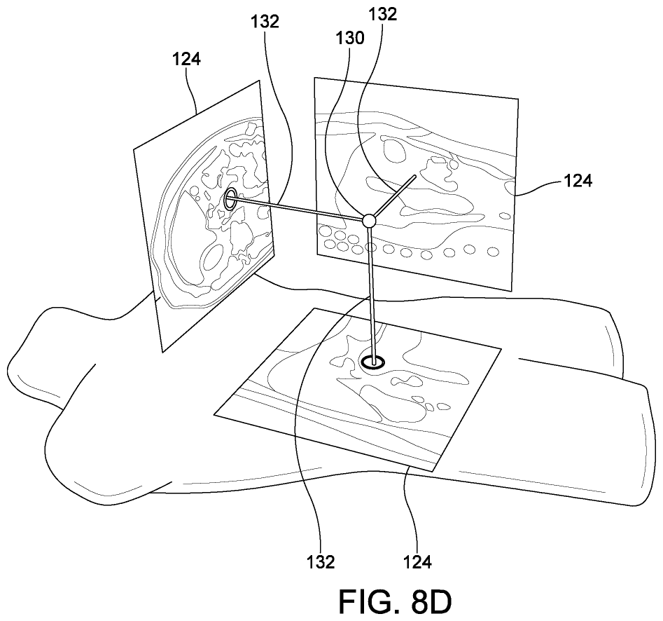

The step 208 can also include identifying target locations in the medical imaging data. As shown in FIGS. 8A-8D, target identification can use multi-planar reconstruction (MPR) visualization, wherein three orthogonal MPR slices 124 can be displayed simultaneously and a user can position the intersection point of the three slices at the target location. Alternatively, target identification can use fingertip-based interaction if the medical imaging data is displayed in the augmented reality system 102, wherein hand tracking enables the user to point to or touch the target location in holographic imaging displays. Multiple targets can be identified for procedures requiring multiple instrument placements.

The method 200 can include a step 210 of establishing a second coordinate transformation between the imaging coordinate system and a patient coordinate system representing the patient's physical anatomy. This second transformation can enable anatomical registration, aligning virtual anatomical content with the patient's physical body. In some embodiments, the second coordinate transformation can be established using a fiducial-based registration approach. External fiducial markers 126 can be positioned on the patient's skin surface before pre-procedural imaging is acquired. The fiducial positions can be identified in the medical imaging data using automatic segmentation algorithms (such as threshold-based detection, blob detection, or AI-based segmentation) or manual identification. During the procedure, the fiducial 126 positions on the patient can be detected using optical detection (cameras or depth sensors detecting fiducials with distinctive optical features), surface scanning (depth sensors or LiDAR capturing the patient's surface including fiducial locations), or a tracked stylus touching each fiducial marker. The system 100 can compute the second coordinate transformation using point-set registration algorithms that find the optimal transformation aligning the corresponding fiducial positions in the imaging data and on the physical patient.

Alternatively, the second coordinate transformation can be established using a markerless surface-based registration approach. The medical imaging data can be processed to extract a three-dimensional surface representing the patient's skin surface using segmentation algorithms. During the procedure, the patient's skin surface can be captured using depth sensors integrated into the augmented reality system 102. The user can scan the patient's skin surface by moving their head to view the patient from multiple angles, with the system accumulating depth data to build a complete surface model. The system 100 can match the captured patient surface to the segmented skin surface using surface-to-surface registration algorithms such as iterative closest point (ICP). The computed transformation can be the second coordinate transformation.

In certain other embodiments, the second coordinate transformation can be established using an ultrasound reference plane registration approach. An ultrasound probe 125 with an attached tracking sensor 114 can be positioned on the patient to acquire a real-time ultrasound image. The tracking system 104 can determine the position and orientation of the ultrasound image plane 128 using the tracking sensor and calibration transformation. The system can identify the corresponding anatomical region in the pre-procedural medical imaging data that matches the ultrasound image plane 128. Image registration algorithms can compare the real-time ultrasound image to the pre-procedural imaging using similarity metrics such as mutual information or cross-correlation. The system 100 can search for the transformation that maximizes similarity between the ultrasound image and the pre-procedural imaging. Once the optimal transformation is found, the second coordinate transformation can be established.

Alternatively, the second coordinate transformation can be established using an anatomical landmark-based registration approach. Distinctive anatomical features (such as bony prominences, skin features, tumors, or other identifiable structures) can be identified in the medical imaging data. During the procedure, the physical locations of the anatomical landmarks on the patient can be determined using a tracked stylus touching the landmarks, or using cameras or depth sensors detecting landmarks with visible features. The system 100 can compute the second coordinate transformation using point-set registration algorithms aligning the landmark positions. Hybrid anatomical registration approaches can combine multiple registration methods. For example, coarse registration can first be performed using surface-based matching, followed by refinement using fiducial-based registration.

The method 200 can include a step 212 of attaching a tracking sensor 114 to a medical instrument 112 to be used in the medical procedure. The medical instrument 112 can include a needle, a trocar, an ultrasound probe, a laparoscopic instrument, a catheter, an ablation probe, or other interventional device. The tracking sensor can be attached using brackets, clips, housings, or adhesive mounts as described hereinabove. The spatial relationship between the tracking sensor 114 and the clinically relevant portion of the instrument 112 can be determined through calibration as described hereinabove.

The method 200 can include a step 214 of determining a position and orientation of the tracked medical instrument 112 using the tracking system 104. The tracking system 104 can determine the position and orientation of the tracking sensor 114 attached to the instrument 112, providing six degrees of freedom (6-DOF) tracking. The tracking system 104 can provide real-time updates at rates of 20 Hz or higher. The calibration transformation can be applied to convert the tracked sensor 114 position to the instrument's functional position (such as needle tip position or ultrasound image plane position).

The method 200 can include a step 216 of transforming the position and orientation of the tracked medical instrument through the first coordinate transformation established in step 206 to determine a display position and orientation in the augmented reality coordinate system. The transformation can be applied as a matrix multiplication, with the result being the instrument position and orientation expressed in augmented reality coordinates specifying where holographic content should be displayed to appear spatially aligned with the physical instrument.

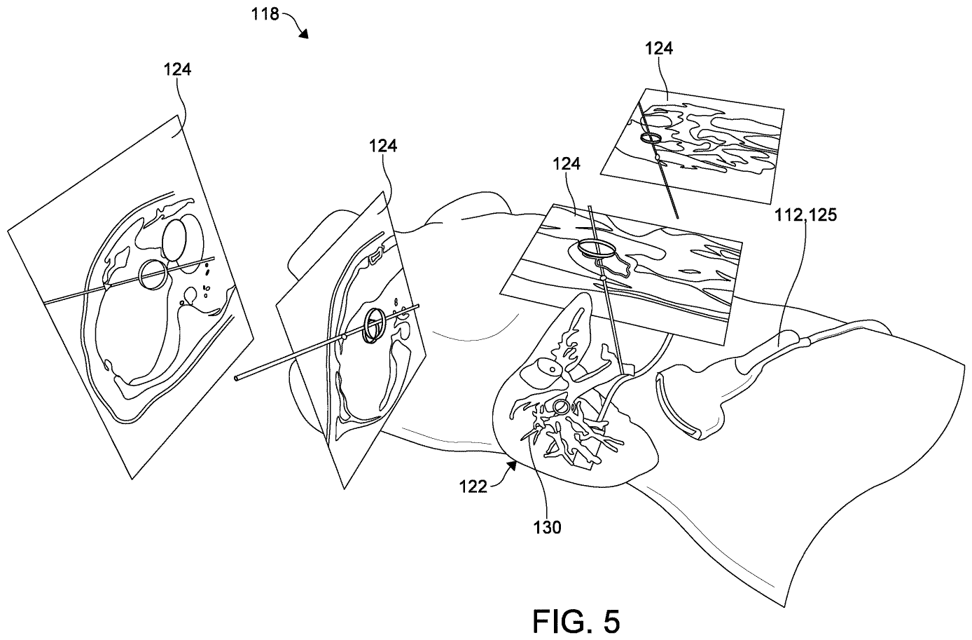

The method 200 can include a step 218 of displaying, using the augmented reality system 102, holographic content 118 derived from the medical imaging data, for example, as shown in FIG. 5. The holographic content can be positioned using the second coordinate transformation established in step 210 such that the holographic content 118 appears spatially aligned with the patient's physical anatomy. The holographic content 118 can include three-dimensional anatomical structures segmented 122 from the medical imaging data, rendered as surface meshes. Multiple anatomical structures 122 can be displayed simultaneously with independent visibility control, adjustable transparency, assigned colors, and selectable rendering styles (solid, wireframe, or point cloud). The holographic content can include two-dimensional imaging slices 124 extracted from the medical imaging data as multi-planar reconstructions (MPR) in arbitrary orientations. The imaging slices 124 can be positioned in three-dimensional space at their corresponding spatial locations relative to the patient's anatomy.

The holographic content can include target markers 130 indicating procedural target locations identified in step 208. Procedural target locations are specific anatomical locations in the medical imaging data representing where the physician intends to navigate a medical instrument, such as a tumor center for biopsy, an ablation zone, a needle insertion endpoint, a lesion requiring treatment, or an anatomical landmark for needle guidance. During step 208, these target locations are identified and marked in the pre-procedural or intra-procedural imaging data using multi-planar reconstruction (MPR) visualization or fingertip-based interaction, creating digital markers that define the three-dimensional coordinates of each target. These digital markers are then displayed during the procedure as holographic target markers 130. A target marker 130 can be represented as a geometric shape (sphere, ellipsoid, crosshair, or bull's-eye) positioned at the target location and rendered with high visibility. Multiple target markers 130 can be displayed with labels or different colors to distinguish them. The appearance of holographic content 118 can be adjusted based on visualization parameters including window and level settings, transparency settings, and color settings.

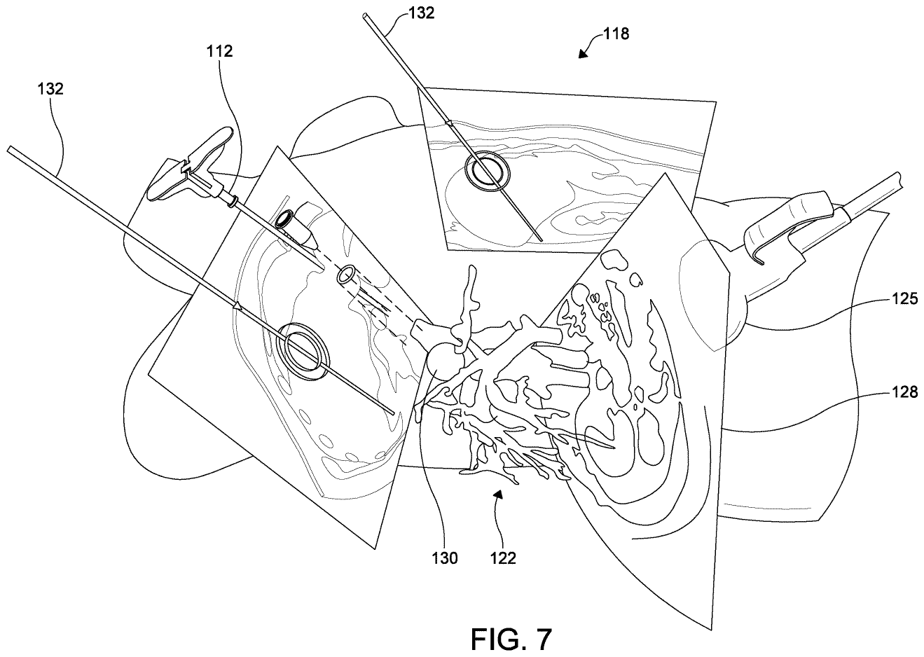

The method 200 can include a step 220 of displaying, using the augmented reality system 102, a virtual representation 116 of the tracked medical instrument 112 at the display position and orientation determined in step 216, for example, as shown in FIG. 7. The virtual representation 116 can appear spatially aligned with the physical instrument 112, providing visual feedback about instrument location even when the physical instrument is partially or fully obscured by the patient's body. The virtual representation 116 can be a three-dimensional model of the instrument, a simplified representation, or a symbolic representation. The virtual representation 116 can be color-coded or visually distinctive to differentiate it from anatomical content.

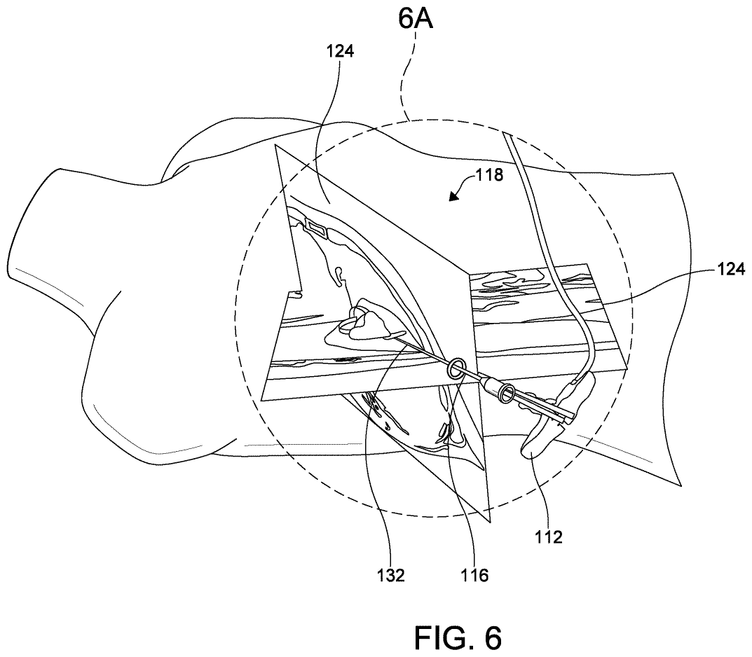

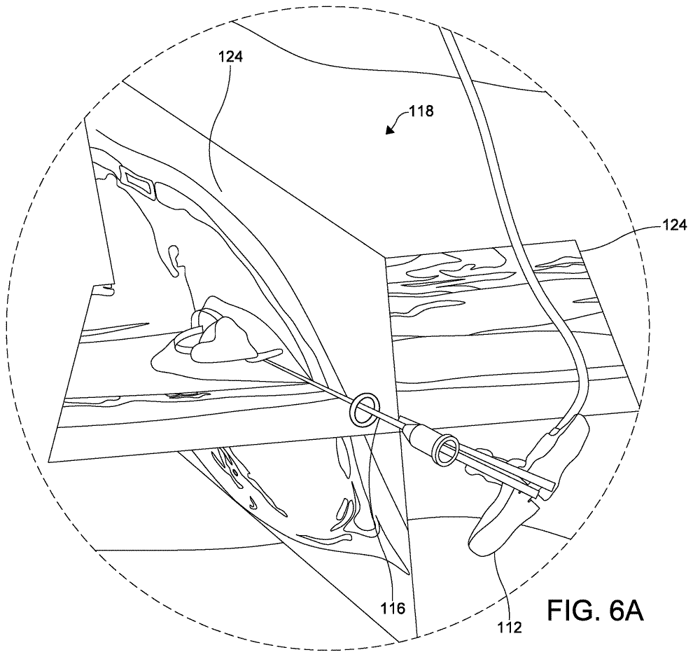

With reference to FIG. 7, the virtual representation 116 can include a Holographic Light Ray (HLR) 132 extending from the instrument tip along the instrument's trajectory, showing the projected path toward a target location. The HLR 132 can be color-coded to provide feedback: green when well-aligned with the target, yellow when slightly misaligned, and red when significantly misaligned. The HLR 132 can display warnings when the projected trajectory approaches critical structures to avoid. Multi-planar reconstruction slices 124 can be positioned to bisect the tracked instrument, for example as shown in FIGS. 6 and 6A. A peri-axial slice can be oriented along the instrument trajectory, and a peri-sagittal slice can be oriented perpendicular to the instrument. The MPR slices 124 can show the target location, current instrument position, and anatomical structures along the path. For tracked ultrasound probes, the virtual representation can include display of the real-time ultrasound image as a holographic plane positioned at the actual ultrasound image plane location.

In some embodiments, the method 200 can include a step 222 of updating the display of the virtual representation in real-time as the tracked medical instrument moves. The tracking system 104 can continuously determine updated instrument positions, and steps 216 and 220 can be repeated to transform the new instrument position to display coordinates and update the displayed virtual representation. The real-time updates can provide smooth, low-latency visualization with total end-to-end latency preferably less than 50 milliseconds.

As the medical instrument 112 moves, guidance elements that depend on the instrument's position can update accordingly. HLR projections 132 can update to show the current trajectory. MPR slices 124 bisecting the instrument can update their positions and orientations to maintain alignment. Distance indicators can update to show current distance from instrument tip to target. The method 200 can continue with real-time tracking and display throughout the medical procedure, enabling navigation of the instrument to the target location while viewing holographic guidance.

FIG. 8A-8D illustrates alternative imaging modality combinations and processing configurations for augmented reality medical guidance, showing a spectrum from comprehensive multi-modal processing to streamlined single-modality workflows. The disclosed systems and methods can accommodate different clinical scenarios, available imaging data, and time constraints.

FIG. 8A depicts a CT+MRI/Fusion+Segmentation configuration representing the most comprehensive approach. Pre-procedural CT imaging and MRI imaging of patient anatomy are both acquired. Image fusion algorithms can combine the CT and MRI data to leverage complementary information from both modalities. Segmentation algorithms can extract anatomical structures 122 from the fused imaging data, creating three-dimensional surface models of organs, vessels, tumors, and other relevant anatomy. Multi-planar reconstruction (MPR) slices 124 can be generated from either the CT data, the MRI data, or the fused data. During the procedure, holographic content 118 can include segmented structures 122 rendered as three-dimensional models, MPR slices 124 showing cross-sectional anatomy, and target markers 130. This configuration can provide maximum anatomical detail and is suitable for complex procedures requiring comprehensive visualization, though it requires the most pre-procedural preparation time.

FIG. 8B depicts a CT+MRI/Fusion configuration with moderate processing requirements. Pre-procedural CT imaging and MRI imaging can both be acquired, and image fusion can combine the data from both modalities. However, segmentation is not performed or is performed only for critical structures. MPR slices 124 can be generated from the fused CT+MRI data. During the procedure, holographic content 118 can include MPR slices 124 showing fused multi-modal imaging and target markers 130, but does not include comprehensive segmented anatomical models. This configuration can provide multi-modal imaging benefits with reduced pre-procedural preparation time compared to panel (a).

FIG. 8C depicts a CT+MRI configuration without fusion or segmentation. Pre-procedural CT imaging and MRI imaging can both be available, but are not fused together. MPR slices 124 can be generated from either the CT data or the MRI data independently. The user can switch between CT-based MPR slices 124 and MRI-based MPR slices 124 during the procedure, or can view both simultaneously. During the procedure, holographic content 118 can include MPR slices 124 from one or both imaging modalities and target markers 130. No segmented structures or fused images are displayed. This configuration can enable multi-modal imaging guidance with minimal pre-procedural processing.

FIG. 8D depicts a CT-only configuration representing the most streamlined approach. Only pre-procedural CT imaging is acquired. No MRI imaging, fusion, or segmentation is performed. MPR slices 124 can be generated from the CT data. During the procedure, holographic content 118 can include MPR slices 124 showing CT anatomy and target markers 130. This configuration can require the least pre-procedural preparation time and is suitable for straightforward procedures where single-modality imaging provides sufficient anatomical information.

Complex oncologic procedures may benefit from panel (a) configuration with comprehensive multi-modal fusion and segmentation. Emergency or time-sensitive procedures may use panel (d) configuration with streamlined single-modality imaging. Interventional radiology procedures may use panel (b) or (c) configurations balancing multi-modal information with workflow efficiency. The system can adapt to the specific clinical scenario, available imaging modalities, time constraints, and procedural complexity, providing flexibility across diverse medical applications.

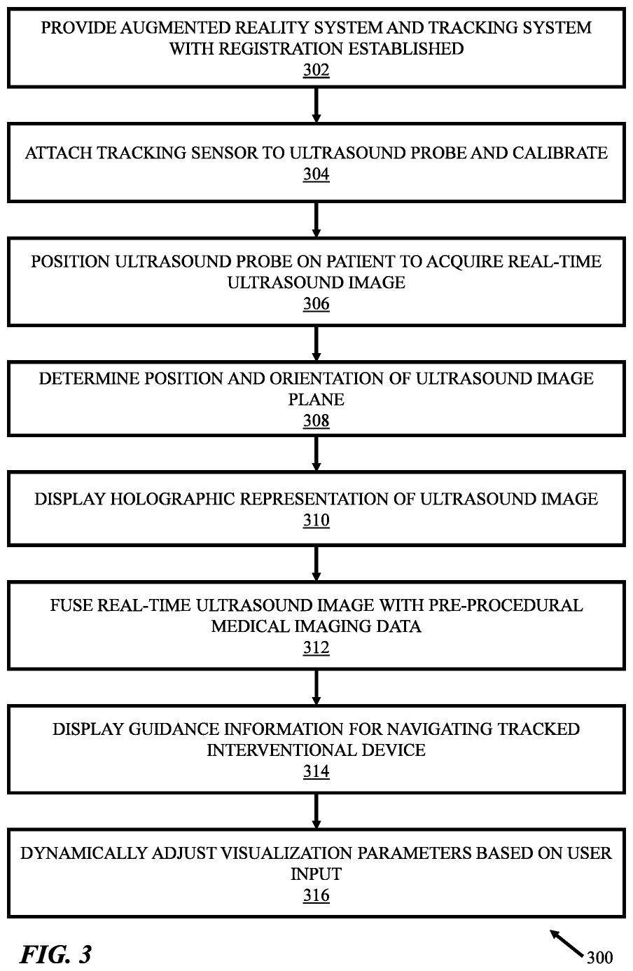

FIG. 3 illustrates a method 300 for providing multi-modal visualization and guidance in an augmented reality medical procedure. The method 300 can enable fusion of real-time ultrasound imaging with pre-procedural imaging and can provide comprehensive guidance information for navigating interventional devices to target locations. The method 300 can include a step 302 of providing an augmented reality system 102 and tracking system 104 with registration established between coordinate systems. The registration can be established using method 200 described above, including device-level registration and anatomical registration. Pre-procedural medical imaging data can be obtained as described in step 208 of method 200.

The method 300 can include a step 304 of attaching a tracking sensor to an ultrasound probe and calibrating the ultrasound probe. Calibration can determine the transformation from the tracking sensor to the ultrasound image plane. The ultrasound probe can be a conventional wired ultrasound probe or a wireless ultrasound probe. For wireless implementations, a wireless tracking sensor can be used, creating a fully wireless tracked probe. The method 300 can include a step 306 of positioning the ultrasound probe on the patient to acquire a real-time ultrasound image of the patient's anatomy. The ultrasound image can show anatomical structures relevant to the procedure, such as a target lesion or tumor, surrounding organs, vessels, or other landmarks.

The method 300 can include a step 308 of determining a position and orientation of the ultrasound image plane 128 using the tracking system 104 and the calibration transformation from step 304. The tracking system 104 can determine the tracking sensor position in the tracking coordinate system. The calibration transformation can convert the sensor position to the ultrasound image plane position and orientation. The image plane position can be transformed to the augmented reality coordinate system using the device-level registration established in step 302.

The method 300 can include a step 310 of displaying, using the augmented reality system 102, a holographic representation of the ultrasound image as a plane positioned in three-dimensional space at the actual location and orientation of the ultrasound image plane. This ultrasound flashlight mode can show the real-time ultrasound image appearing to “slice through” the patient's body at the probe location. The ultrasound image can be texture-mapped onto the holographic plane, with the plane positioned and oriented to match the physical ultrasound image plane position.

The method 300 can include a step 312 of fusing the real-time ultrasound image with the pre-procedural medical imaging data from step 302. The fusion can combine complementary information from the two imaging modalities. The fusion can be implemented in an ultrasound-only mode, wherein only the real-time ultrasound image is displayed without overlay of pre-procedural imaging, providing ultrasound flashlight visualization without fusion. Alternatively, the fusion can be implemented in a texture overlay mode. The system 100 can determine which region of the pre-procedural imaging corresponds to the current ultrasound image plane position and orientation. A texture extracted from that region of the pre-procedural imaging can be displayed on the holographic plane along with or instead of the ultrasound image. The user can toggle between showing the ultrasound texture, the pre-procedural imaging texture, or a blended combination using adjustable alpha blending.

Alternatively, the fusion can be implemented in an edge map fusion mode. Edges can be extracted from both the ultrasound image and the pre-procedural imaging using edge detection algorithms. The edges from both sources can be displayed simultaneously, allowing visualization of structure boundaries from both imaging modalities. The edges can be color-coded to distinguish between sources (such as ultrasound edges in green and CT edges in blue). Edge detection thresholds can be independently adjusted for each modality.

Alternatively, the fusion can be implemented in a side-by-side mode, wherein the ultrasound image and the corresponding region from pre-procedural imaging are displayed as separate holographic panels positioned adjacent to each other, enabling direct comparison without overlap. The user can switch between fusion modes during the procedure using voice commands, hand gestures, or controller input. Voice commands can include “show ultrasound only,” “show CT overlay,” “show edge fusion,” or “show side-by-side.”

The method 300 can include a step 314 of displaying guidance information assisting navigation of a tracked interventional device to a target location. A tracking sensor can be attached to the interventional device as described in step 212 of method 200, and the device position can be determined as described in step 214 of method 200. The guidance information can include a virtual representation of the tracked interventional device displayed at the device's actual position and orientation. The virtual device can be a three-dimensional model, simplified representation, or symbolic representation as described in step 220 of method 200.

In some embodiments, with reference to FIG. 3, the guidance information can include the Holographic Light Ray (HLR) 132 extending from the interventional device 112 along its trajectory toward the target location. The guidance information can include multi-planar reconstruction (MPR) slices 124 positioned to bisect the tracked device 112. A peri-axial slice can provide a view along the device trajectory, and a peri-sagittal slice can provide a perpendicular view. The MPR slices 124 can update in real-time as the device moves, maintaining alignment with the current device orientation. The guidance information can include the target marker 130 indicating the three-dimensional position of the procedural target.

The method 300 can include a step 316 of dynamically adjusting visualization parameters based on user input during the procedure. The user can control what content is displayed and how it appears. Window and level settings can be adjusted to modify brightness and contrast of displayed imaging data. Window and level can be optimized for different tissue types (such as bone window, soft tissue window, or lung window for CT imaging). Preset window/level values can be selected by voice command, or values can be adjusted continuously using hand gestures or controller input. Edge detection threshold settings can control the sensitivity of edge detection algorithms. Increasing the threshold can show only strong edges, while decreasing the threshold can show more subtle edges. The threshold can be adjusted independently for different imaging modalities. Threshold adjustment can use voice commands specifying increase or decrease, or can use gestures.

Depth settings can control how much content is displayed. A maximum depth parameter can limit display to structures within a certain distance from the viewer or from a reference plane, reducing visual clutter. Depth settings can be adjusted to “peel away” outer structures to reveal deeper anatomy. Layer visibility toggles can show or hide specific content types or anatomical structures. The user can speak commands such as “show skin,” “hide skin,” “show vessels,” “hide bone,” or “show target” to control visibility. Each segmented anatomical structure and each guidance element can have independent visibility control. Transparency settings can adjust the opacity of displayed content. Structures can be made more transparent to see through them to underlying anatomy, or more opaque for better visibility. Transparency can be adjusted for each content layer independently. Target location adjustments can enable repositioning of target markers during the procedure. The user can issue voice commands such as “move target up 5 millimeters,” “move target left,” or “move target to device tip” to adjust the target position. The target can also be moved using hand gestures, such as pinching the target marker and dragging it to a new location. The method 300 can continue with real-time ultrasound imaging, fusion visualization, and guidance display throughout the procedure, enabling the physician to navigate the interventional device to the target with multi-modal imaging guidance.

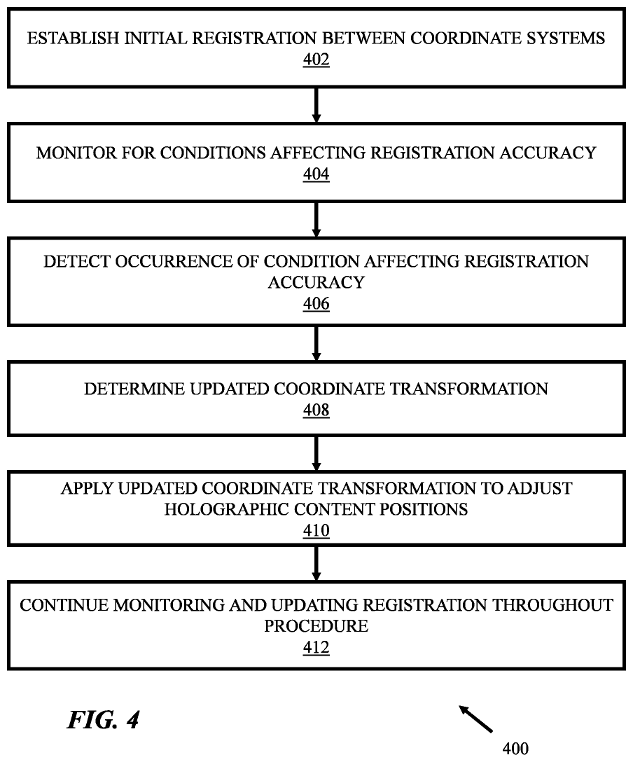

FIG. 4 illustrates a method 400 for dynamically maintaining registration accuracy during an augmented reality medical procedure despite changing conditions. The method 400 can detect and compensate for patient motion, table movement, respiratory motion, and anatomical deformation. The method 400 can include a step 402 of establishing initial registration between coordinate systems using method 200 described above. The initial registration can include device-level registration (step 206) and anatomical registration (step 210). A medical procedure can begin with holographic guidance displayed based on the initial registration. The method 400 can include a step 404 of monitoring for conditions that can affect registration accuracy during the procedure. Monitored conditions can include patient motion, table movement, respiratory motion, and anatomical deformation.

The method 400 can include a step 406 of detecting occurrence of a condition affecting registration accuracy. Patient motion can be detected by tracking the position of a reference attached to or associated with the patient. An Anatomical Registration Device (XRD) can be positioned on the patient's body and can include one or more tracking sensors 114. The tracking system 104 can continuously determine the XRD position and orientation throughout the procedure. Gross patient motion can be detected when the XRD position changes beyond a threshold amount. Alternatively, patient motion can be detected using anatomical landmark tracking or surface tracking when an XRD is not present. Specific anatomical features visible to cameras or depth sensors in the augmented reality system 102 can be tracked. The patient's skin surface can be tracked using depth sensors. Position changes of landmarks or surfaces can indicate patient motion.

Table movement can be detected by tracking a reference attached to the procedure table. A registration device positioned on the table surface or attached to a table rail can be tracked. If the registration device position changes relative to a room coordinate system tracked by the augmented reality system's room tracking, table movement has occurred. Respiratory motion can be detected by tracking markers or surface features that move with respiration. A sensor positioned on the patient's chest or abdomen can detect respiratory motion. An XRD positioned on the chest or abdomen can provide respiratory motion tracking. Surface tracking of the chest or abdominal surface can detect respiratory motion. Ultrasound imaging of the diaphragm can detect respiratory phase. Anatomical deformation can be detected by comparing real-time imaging to pre-procedural imaging. When a tracked ultrasound probe acquires images, the real-time ultrasound images can be compared to corresponding regions in pre-procedural imaging. If anatomical structures visible in ultrasound do not align well with structures in pre-procedural imaging even after attempting registration, deformation has likely occurred. Image similarity metrics (such as mutual information or cross-correlation) can quantitatively assess alignment quality. If similarity falls below a threshold, significant deformation is likely present.

The method 400 can include a step 408 of determining an updated coordinate transformation that compensates for the detected condition. For patient motion, the system can compute an updated coordinate transformation by determining the translational and rotational displacement of the XRD (or tracked landmarks or surface) from its position at the time of initial registration to its current position. The updated transformation can apply the same displacement to the holographic content, causing the holographic content to move with the patient.

For table movement when the patient and table move together, the registration update can treat the movement similarly to patient motion, adjusting holographic content to move with the table and patient. If the electromagnetic field generator 106 is mounted to the table, the generator moves with the table and device-level registration can remain valid. If the generator does not move with the table, both device-level registration and anatomical registration can require updating.

For respiratory motion, various compensation approaches can be used. Respiratory gating can identify specific respiratory phases (such as end-expiration) and can display holographic content only during those phases or can inform the user of the current respiratory phase for timing instrument manipulation. Alternatively, dynamic respiratory motion compensation can continuously adjust holographic content positions to follow respiratory motion. The system can model the respiratory cycle based on observed motion patterns and can predict anatomy position at any point in the cycle.

For anatomical deformation, when significant deformation is detected, compensation approaches can include alerting the user that registration accuracy may be degraded, transitioning to real-time imaging guidance using ultrasound rather than pre-procedural imaging overlays, or applying non-rigid registration algorithms to deform the pre-procedural imaging to match current anatomy. Non-rigid registration can compute a deformation field describing how each point in the pre-procedural imaging has moved.

The method 400 can include a step 410 of applying the updated coordinate transformation to adjust positions of displayed holographic content. The holographic content positions can be updated to maintain correct spatial alignment with the patient's anatomy despite the changing conditions. Real-time registration updates can be applied continuously or at regular intervals. The holographic content positions can be updated smoothly to avoid jarring visual discontinuities. Interpolation or filtering can smooth transitions when applying registration updates. For respiratory gating, holographic content can be displayed or hidden based on the current respiratory phase. For dynamic respiratory compensation, holographic content positions can be continuously adjusted to follow the predicted anatomy motion throughout the respiratory cycle. For anatomical deformation compensation, the system can display alerts informing the physician of degraded registration accuracy. The visualization mode can be switched to emphasize real-time ultrasound guidance. If non-rigid registration is applied, the deformed pre-procedural imaging can be displayed instead of the original rigid registration.

The method 400 can include a step 412 of continuing to monitor and update registration throughout the procedure. Steps 404-410 can be repeated as needed to maintain registration accuracy. Registration quality metrics can be monitored to detect when registration accuracy degrades, and the system can alert the user and can suggest or automatically perform re-registration when needed. Automatic registration verification can periodically check registration accuracy using available information. If an XRD or registration device position is known from both imaging and tracking, the distance between expected and actual positions can verify registration accuracy. If anatomical landmarks are visible in real-time ultrasound and in pre-procedural imaging, their alignment can verify anatomical registration. The method 400 can provide continuous dynamic registration maintenance throughout the medical procedure, ensuring that holographic guidance remains accurately aligned with the patient's anatomy despite patient motion, respiratory motion, table movement, or anatomical deformation.

### EXAMPLES

The following examples illustrate complete procedural workflows using the systems 100 and methods 200, 300, 400 described above. These examples demonstrate how the flexible registration framework, multi-modal visualization capabilities, and dynamic compensation methods can be applied to different clinical scenarios. Each example shows specific choices of registration approaches, tracking modalities, and visualization modes selected based on procedural requirements and available equipment. The examples are illustrative and non-limiting; the disclosed systems and methods can be applied to numerous other medical procedures and clinical applications beyond those specifically described.

### Example 1: Liver Biopsy With Manual Registration and Fiducial-Based Anatomical Registration

A patient requires a percutaneous biopsy of a liver lesion identified on pre-procedural imaging. Pre-procedural CT imaging of the abdomen is acquired showing the liver and a suspicious lesion. Three adhesive fiducial markers 126 are placed on the patient's skin surface in the right upper quadrant around the planned biopsy site before the CT scan is performed, ensuring the fiducials appear in the imaging data. The CT DICOM data is loaded into the augmented reality guidance system 100. Automatic segmentation is performed to extract the liver surface as a segmented structure 122, the lesion, and the three fiducial markers 126. The target location indicated by target marker 130 within the lesion is identified using MPR tri-section visualization, positioning the intersection of axial, sagittal, and coronal MPR slices 124 at the desired biopsy location.

On the day of the procedure, a planar electromagnetic field generator 106 is positioned on the procedure table beneath the patient. The physician wears a head-mounted display device (part of augmented reality system 102). Device-level registration is performed using the manual registration approach (step 206 of method 200). A virtual holographic representation of the field generator 106 is displayed in the augmented reality view. The physician uses hand gestures to manipulate the holographic generator position and orientation until it visually aligns with the physical field generator 106. Alignment indicators change from red to yellow to green as alignment improves. The physician provides voice confirmation “confirm alignment,” and the system computes and stores the first coordinate transformation. Anatomical registration is performed using the fiducial-based approach (step 210 of method 200). The three fiducial markers 126 on the patient's skin are detected using the depth sensors in the head-mounted display device, which capture the patient's surface including the fiducial locations. The system automatically matches the detected fiducial positions to the fiducial positions identified in the CT data, computing the second coordinate transformation using least-squares point-set registration. Registration quality metrics indicate good alignment with mean fiducial registration error of 2.1 mm.

A biopsy needle (medical instrument 112) with a 6-DOF electromagnetic sensor 114 attached to the needle hub via a clip bracket (tracking sensor 114) is prepared. The calibration parameters for this sensor-needle combination are retrieved from the system database, establishing the offset from sensor 114 to needle tip. The physician positions the needle at the skin entry point. Through the head-mounted display, the physician views holographic content 118 including the segmented liver 122 rendered in semi-transparent orange, the lesion rendered in solid red, and the target marker 130 displayed as a yellow sphere at the target location. Virtual needle representation 116 appears at the actual needle position. HLR 132 extends from the needle tip along the needle trajectory toward the target marker 130, initially displayed in red indicating poor alignment.

The physician adjusts the needle angle while viewing the guidance. As alignment improves, HLR 132 changes to yellow and then to green. Peri-axial and peri-sagittal MPR slices 124 bisect the virtual needle 116, showing CT cross-sections along and perpendicular to the needle path. A distance indicator shows “Distance to target: 47 mm.” The physician advances the needle while viewing the real-time guidance. Virtual needle representation 116 updates smoothly as the physical needle advances. The distance indicator counts down: “35 mm . . . 22 mm . . . 11 mm . . . 3 mm.” When the needle tip reaches the target, HLR 132 terminates at the target marker 130 and the distance indicator shows “At target.” The physician acquires the biopsy sample. Histopathology confirms diagnostic tissue was obtained.

### Example 2: Kidney Ablation With Automated Registration, Ultrasound Reference Plane Registration, and Multi-Modal Fusion

A patient requires radiofrequency ablation of a small renal cell carcinoma in the left kidney. Pre-procedural contrast-enhanced MRI shows the kidney, tumor, and surrounding vessels. The kidney, tumor (target lesion), and renal vessels are segmented from the MRI as segmented structures 122. The target location indicated by target marker 130 is identified at the center of the tumor using fingertip-based target selection in the augmented reality planning interface. A tabletop electromagnetic field generator 106 is positioned on the procedure table. The physician wears a head-mounted display device (part of augmented reality system 102) with a wireless electromagnetic sensor 108 attached to the top of the headset using an adhesive mount (functioning as tracking sensor 114 for the headset). Device-level registration is performed using the automated registration approach (step 206 of method 200). The wireless electromagnetic sensor 108 position is continuously tracked by the electromagnetic tracking system 104 in tracking coordinates, while the head-mounted display device knows the sensor position in augmented reality coordinates based on the mounting location. The system automatically computes the first coordinate transformation without requiring manual alignment. The automated registration provides continuous updates throughout the procedure.

A wireless ultrasound probe 125 with an attached wireless electromagnetic sensor 108 (functioning as tracking sensor 114 for the probe, creating a fully wireless tracked probe) is used for real-time imaging. The probe-sensor combination was previously calibrated, with calibration parameters stored and automatically retrieved. Anatomical registration is performed using the ultrasound reference plane registration approach (step 210 of method 200). The physician positions the ultrasound probe 125 on the patient's flank to image the kidney and tumor. The real-time ultrasound image plane 128 is displayed in the head-mounted display as the ultrasound flashlight (step 310 of method 300).

The system 100 performs image registration comparing the real-time ultrasound image plane 128 to the MRI data, using mutual information as the similarity metric to accommodate the different imaging modalities. The optimization algorithm searches for the transformation that maximizes alignment between the ultrasound and MRI. Once optimal alignment is found, the second coordinate transformation T2 is established, relating the MRI coordinate system to the patient's current anatomy without requiring external fiducial markers 126. Registration quality metrics indicate successful registration with normalized mutual information value of 0.68. The physician uses multi-modal fusion visualization (method 300) while navigating the ablation probe (medical instrument 112) to the target marker 130. The ultrasound image plane 128 displays edge map fusion mode (step 312), showing ultrasound-detected edges in green and MRI-detected edges in blue. The physician can see both the real-time anatomy (ultrasound) and the pre-procedural vascular anatomy (MRI vessels as segmented structures 122) simultaneously. Voice command “show texture overlay” switches to texture overlay mode, blending the ultrasound and MRI textures with adjustable weighting.

An ablation probe (medical instrument 112) with attached electromagnetic sensor 108 (tracking sensor 114) is tracked. Virtual probe representation 116 and HLR 132 provide trajectory guidance toward target marker 130. Peri-axial and peri-sagittal MPR slices 124 show both MRI and ultrasound data, providing comprehensive visualization. A distance indicator shows remaining distance to target. During the procedure, the patient's breathing causes respiratory motion. The system implements method 400 for dynamic compensation. The wireless electromagnetic sensor 108 on the patient's chest (the ultrasound probe 125 positioned on the flank) detects respiratory motion. The system models the respiratory cycle and implements dynamic respiratory motion compensation (step 408), continuously adjusting the holographic content 118 positions to follow the predicted kidney motion throughout the respiratory cycle. The physician can operate continuously rather than waiting for specific respiratory phases. The physician advances the ablation probe (medical instrument 112) to the target marker 130 using the multi-modal guidance. Once positioned, radiofrequency ablation is performed. Post-ablation ultrasound and subsequent follow-up imaging confirm complete tumor treatment with preservation of surrounding vessels.

### Example 3: Laparoscopic Partial Hepatectomy With Optical Registration and Surface-Based Anatomical Registration

A patient undergoes laparoscopic partial liver resection for a liver tumor. Pre-procedural contrast-enhanced CT shows liver anatomy, hepatic vessels, portal vein branches, hepatic veins, and the tumor. The liver, major vessels, and tumor are segmented as segmented structures 122. The surgical plan includes resection margins defined in the imaging, creating a resection zone boundary. An articulating arm-mounted electromagnetic field generator 106 is positioned to provide optimal working volume over the patient's abdomen. Printed image targets (optical markers 120) are attached to the field generator 106. The surgeon wears a head-mounted display device (part of augmented reality system 102). Device-level registration is performed using the optical registration approach (step 206 of method 200). Cameras in the head-mounted display device detect the image targets (optical markers 120) on the field generator 106 and compute the generator position and orientation in augmented reality coordinates. The system computes the first coordinate transformation T1 from the known positions in both coordinate systems.

No external fiducial markers 126 are placed on the patient. Instead, anatomical registration is performed using the surface-based registration approach (step 210 of method 200). Before insufflation, the surgeon scans the patient's abdominal surface by moving their head to view the abdomen from multiple angles. The head-mounted display depth sensors capture the skin surface, building a three-dimensional surface model. The captured surface is matched to the segmented skin surface 122 from the CT using iterative closest point (ICP) surface registration. The second coordinate transformation T2 is computed. Registration quality metrics show mean surface alignment error of 3.2 mm.

Before trocar insertion, the surgeon views holographic content 118 displayed through the head-mounted display device, with the holographic content 118 appearing spatially positioned at the surgical site and superimposed on the patient external abdominal surface. The holographic content 118 includes the liver and vessels (segmented structures 122) and tumor appearing to float within or above the patient body at their actual anatomical locations. This pre-insufflation visualization guides optimal port placement. The surgeon can see where the tumor is located beneath the skin surface and can plan trocar positions to provide optimal instrument access while avoiding vessel injury. After insufflation and trocar placement, the surgeon inserts a laparoscopic ultrasound probe 125 with attached electromagnetic sensor 108 (tracking instrument sensor 114). The probe is calibrated for sensor-to-image-plane transformation. The surgeon uses the ultrasound flashlight mode (method 300) to visualize real-time intra-operative anatomy via ultrasound image plane 128. Because insufflation has caused organ displacement (anatomical deformation), the system implements method 400 for deformation compensation.

Comparison of the real-time laparoscopic ultrasound image plane 128 to the pre-procedural CT reveals misalignment due to deformation (step 406 of method 400). Image similarity metrics fall below threshold, and a warning indicator alerts the surgeon that pre-procedural imaging may not accurately represent current anatomy. The system transitions to ultrasound-based guidance (step 408), emphasizing the real-time ultrasound visualization rather than CT overlays. The surgeon uses ultrasound flashlight to identify vessel locations and tumor boundaries in the current deformed state. During dissection, the surgeon views both the laparoscopic video monitor and the augmented reality holographic content 118 through the head-mounted display device. The holographic overlays show vessel locations (segmented structures 122) and resection margins, providing three-dimensional spatial context that complements the two-dimensional laparoscopic video view. The natural hand-eye coordination enabled by viewing the patient directly (rather than viewing only a video monitor) improves ergonomics.

The surgeon uses voice commands (step 316 of method 300) to control visualization during the procedure: “hide skin” removes the skin surface overlay; “show vessels” ensures hepatic vessels (segmented structures 122) are visible; “adjust transparency” makes the liver semi-transparent to see deeper structures. As dissection progresses, “move target left 5 millimeters” adjusts a target marker 130 position based on intra-operative findings. The resection is completed successfully with negative margins confirmed by intra-operative ultrasound and subsequent histopathology. All major vessels are preserved. The surgeon reports that the augmented reality guidance improved spatial orientation and confidence in margin adequacy during the laparoscopic procedure.

### Example 4: Spinal Needle Placement With Stylus-Based Device Registration and Landmark-Based Anatomical Registration

A patient requires a lumbar puncture at the L3-L4 interspace. Pre-procedural CT of the lumbar spine shows bony anatomy including vertebral bodies, spinous processes, and intervertebral spaces. The vertebrae and skin surface are segmented as segmented structures 122. Anatomical landmarks including the L3 and L4 spinous processes are identified in the imaging. The target location indicated by target marker 130 is identified at the midline in the L3-L4 interspace. A small portable electromagnetic field generator 106 is positioned adjacent to the patient in the prone position. The physician wears a head-mounted display device (part of augmented reality system 102). Device-level registration is performed using the stylus-based registration approach (step 206 of method 200). A tracked stylus with electromagnetic sensor 108 (tracking sensor 114) and calibrated tip is used to touch four corner positions on the field generator 106. The system records the three-dimensional positions in tracking coordinates. The physician also identifies the same four corner locations in the augmented reality display by pointing with a finger (detected by hand tracking). The system computes the first coordinate transformation T1 using point-set registration of the corresponding locations.

Anatomical registration is performed using the landmark-based approach (step 210 of method 200). The physician palpates the L3 and L4 spinous processes on the patient's back and uses the tracked stylus with electromagnetic sensor 108 (tracking sensor 114) to touch the tip to each palpated landmark. The system records the landmark positions in tracking coordinates (converted to AR coordinates using T1). The system computes the second coordinate transformation T2 by matching the touched landmark positions to the landmark positions identified in the CT imaging coordinate system. No external fiducial markers 126 or surface scanning is required.

A spinal needle (medical instrument 112) with electromagnetic sensor 108 (tracking sensor 114) attached to the hub is prepared. The physician positions the needle at the planned skin entry point. Through the head-mounted display device, the physician views holographic content 118 including the vertebrae (segmented structures 122) rendered semi-transparently, showing the bony anatomy beneath the skin. The target marker 130 appears as a sphere in the L3-L4 interspace. Virtual needle representation 116 and HLR 132 provide trajectory guidance. Peri-axial MPR slice 124 along the needle trajectory shows the intervertebral space and confirms the needle is aligned to avoid bone contact. The physician advances the needle while viewing the guidance. As the needle advances, the virtual representation 116 shows progression through tissue layers toward the target marker 130 in the subarachnoid space. The successful dural puncture is confirmed by cerebrospinal fluid return, and the procedure is completed. The augmented reality guidance enabled accurate needle placement on the first attempt, reducing patient discomfort and procedure time.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method for augmented reality medical guidance, comprising:
providing an augmented reality system including a display device;
providing a tracking system capable of determining position and orientation of tracked objects in a tracking coordinate system;
establishing a first coordinate transformation between the tracking coordinate system and an augmented reality coordinate system of the augmented reality system;
obtaining medical imaging data of an anatomy of a patient, the medical imaging data defining an imaging coordinate system;
establishing a second coordinate transformation between the imaging coordinate system and a patient coordinate system representing a physical anatomy of the patient;
determining a position and orientation of the medical instrument using a tracking sensor and the tracking system;
transforming the position and orientation of the medical instrument through the first coordinate transformation to determine a display position in the augmented reality coordinate system;
displaying, using the augmented reality system, holographic content derived from the medical imaging data, the holographic content positioned using the second coordinate transformation to appear spatially aligned with the physical anatomy of the patient; and
displaying, using the augmented reality system, a virtual representation of the medical instrument at the display position.

2. The method of claim 1, wherein establishing the first coordinate transformation comprises:
displaying a virtual holographic representation of a component of the tracking system in the augmented reality system;
receiving user input manipulating a position and orientation of the virtual holographic representation until the virtual holographic representation aligns with a physical component of the tracking system; and
computing the first coordinate transformation based on the aligned position and orientation.

3. The method of claim 1, wherein establishing the first coordinate transformation comprises:
attaching a second tracking sensor to the head-mounted display device;
determining a position of the second tracking sensor in the tracking coordinate system;
determining a position of the second tracking sensor in the augmented reality coordinate system based on a mounting location on the head-mounted display device; and
automatically computing the first coordinate transformation based on simultaneous position measurements in both coordinate systems.

4. The method of claim 1, wherein establishing the second coordinate transformation comprises:
positioning fiducial markers on the patient's skin surface before acquiring the medical imaging data;
identifying positions of the fiducial markers in the medical imaging data;
detecting positions of the fiducial markers on the patient during a medical procedure; and
computing the second coordinate transformation using point-set registration algorithms that align the fiducial marker positions.

5. The method of claim 1, wherein establishing the second coordinate transformation comprises:
extracting a three-dimensional surface representing the patient's skin surface from the medical imaging data;
capturing the patient's skin surface using depth sensors integrated into the augmented reality system during a medical procedure; and
computing the second coordinate transformation using surface-to-surface registration algorithms that match the captured patient surface to the extracted skin surface.

6. The method of claim 1, wherein establishing the second coordinate transformation comprises:
providing an ultrasound probe with an attached ultrasound tracking sensor;
positioning the ultrasound probe on the patient to acquire a real-time ultrasound image;
determining a position and orientation of an ultrasound image plane using the ultrasound tracking sensor;
comparing the real-time ultrasound image to the medical imaging data using image registration algorithms; and
computing the second coordinate transformation based on a transformation that maximizes similarity between the real-time ultrasound image and the medical imaging data.

7. The method of claim 1, wherein the virtual representation of the medical instrument includes a holographic light ray extending from a tip of the medical instrument along a trajectory of the medical instrument toward a target location, the holographic light ray color-coded to indicate alignment status with the target location.

8. The method of claim 1, wherein displaying the holographic content comprises displaying multi-planar reconstruction slices positioned to bisect the medical instrument, the slices showing cross-sectional anatomy along and perpendicular to an instrument trajectory.

9. A method for multi-modal visualization and guidance in an augmented reality medical procedure, comprising:
providing an augmented reality system and a tracking system with registration established between a tracking coordinate system, an augmented reality coordinate system, and an imaging coordinate system of pre-procedural medical imaging data;
attaching a tracking sensor to an ultrasound probe;
positioning the ultrasound probe on a patient to acquire a real-time ultrasound image;
determining a position and orientation of an ultrasound image plane using the tracking sensor;
displaying, using the augmented reality system, a holographic representation of the ultrasound image as a plane positioned in three-dimensional space at an actual location and orientation of the ultrasound image plane;
fusing the real-time ultrasound image with the pre-procedural medical imaging data; and
displaying guidance information for navigating a tracked interventional device to a target location.

10. The method of claim 9, wherein fusing the real-time ultrasound image with the pre-procedural medical imaging data comprises:
determining a region of the pre-procedural medical imaging data corresponding to the ultrasound image plane position and orientation;
extracting a texture from the region of the pre-procedural medical imaging data; and
displaying a blended combination of the real-time ultrasound image and the extracted texture on the holographic plane.

11. The method of claim 9, wherein fusing the real-time ultrasound image with the pre-procedural medical imaging data comprises:
extracting edges from the real-time ultrasound image using edge detection algorithms;
extracting edges from the pre-procedural medical imaging data corresponding to the ultrasound image plane;
displaying the ultrasound edges and the pre-procedural imaging edges simultaneously on the holographic plane; and
color-coding the edges to distinguish between the real-time ultrasound image and the pre-procedural medical imaging data.

12. The method of claim 9, wherein displaying guidance information comprises:
displaying a virtual representation of the tracked interventional device at an actual position and orientation of the tracked interventional device;
displaying a holographic light ray extending from the tracked interventional device toward the target location, the holographic light ray color-coded based on alignment with the target location; and
displaying multi-planar reconstruction slices positioned to bisect the tracked interventional device.

13. The method of claim 9, further comprising dynamically adjusting visualization parameters based on user input during the medical procedure, the visualization parameters including window and level settings, edge detection threshold settings, depth settings, layer visibility toggles, and transparency settings.

14. The method of claim 13, wherein dynamically adjusting visualization parameters comprises receiving voice commands from a user to control what content is displayed and how the content appears.

15. The method of claim 9, further comprising:
receiving user input to reposition the target location during the medical procedure; and
updating a display of a target marker to reflect the repositioned target location.

16. A method for dynamically maintaining registration accuracy during an augmented reality medical procedure, comprising:
establishing initial registration between a tracking coordinate system, an augmented reality coordinate system, and an imaging coordinate system;
displaying holographic content based on the initial registration, the holographic content appearing spatially aligned with a patient's physical anatomy;
monitoring for conditions affecting registration accuracy during the medical procedure;
detecting occurrence of a condition affecting registration accuracy;
determining an updated coordinate transformation that compensates for the detected condition; and
applying the updated coordinate transformation to adjust positions of the displayed holographic content to maintain spatial alignment with the patient's physical anatomy.

17. The method of claim 16, wherein:
the condition affecting registration accuracy including patient motion;
detecting occurrence of the condition comprises tracking a position of an anatomical registration device attached to the patient and detecting when the position changes beyond a threshold amount; and
determining the updated coordinate transformation including computing a displacement of the anatomical registration device from an initial position to a current position and applying the displacement to the holographic content.

18. The method of claim 16, wherein:
the condition affecting registration accuracy comprises respiratory motion;
detecting occurrence of the condition including tracking a marker that moves with respiration; and
determining the updated coordinate transformation including modeling a respiratory cycle and continuously adjusting holographic content positions to follow predicted anatomy motion throughout the respiratory cycle.

19. The method of claim 16, wherein:
the condition affecting registration accuracy including anatomical deformation;
detecting occurrence of the condition comprises comparing a real-time ultrasound image to the pre-procedural imaging and determining that anatomical structures do not align; and
determining the updated coordinate transformation including transitioning to ultrasound-based guidance.

20. The method of claim 16, further comprising:
continuing to monitor and update registration throughout the medical procedure;
monitoring registration quality metrics to detect when registration accuracy degrades; and
alerting a user when registration accuracy degrades below a threshold.
